(19) United States
(12) Patent Application Publication
(10) Pub. No.: US 2023/0123757 A1
(43) Pub. Date: Apr. 20, 2023

(54) COMPOUND FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Hanill LEE, Suwon-si (KR); Hyung Sun KIM, Suwon-si (KR); Sung-Hyun JUNG, Suwon-si (KR); Seonyeong GWAK, Suwon-si (KR); Wook KIM, Suwon-si (KR); Jaehoon KIM, Suwon-si (KR); Chang Ju SHIN, Suwon-si (KR); Dongkyu RYU, Suwon-si (KR); Mijin LEE, Suwon-si (KR); Yoonman LEE, Suwon-si (KR); Suheon IM, Suwon-si (KR)

(21) Appl. No.: 17/961,772

(22) Filed: Oct. 7, 2022

(30) Foreign Application Priority Data
Oct. 8, 2021 (KR) 10-2021-0134190

(51) Int. Cl. H01L 51/00; C07D 403/14; C09K 11/06; C07D 405/14

(52) U.S. Cl. CPC H01L 51/0072; C07D 403/14; C09K 11/06; H01L 51/0067; C07D 405/14; H01L 51/0073; H01L 51/5012

(57) ABSTRACT

A compound for an organic optoelectronic device, an organic optoelectronic device, and a display device, the compound being represented by Chemical Formula 1:

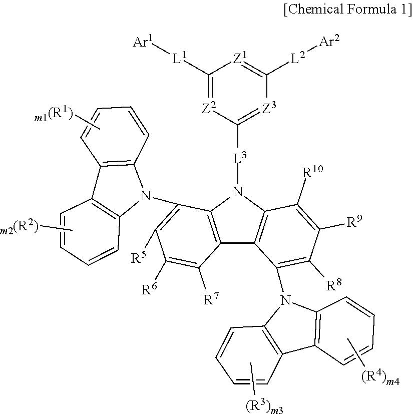

### CROSS-REFERENCE TO RELATED APPLICATION

This application claims priority to and the benefit of Korean Patent Application No. 10-2021-0134190 filed in the Korean Intellectual Property Office on Oct. 8, 2021, the entire contents of which are incorporated herein by reference.

### BACKGROUND

### 1. Field

Embodiments relate to a compound for an organic optoelectronic device, an organic optoelectronic device, and a display device.

### 2. Description of the Related Art

An organic optoelectronic device (e.g., organic optoelectronic diode) is a device that converts electrical energy into photoenergy, and vice versa.

An organic optoelectronic device may be classified as follows in accordance with its driving principles. One is a photoelectric device where excitons generated by photoenergy are separated into electrons and holes and the electrons and holes are transferred to different electrodes respectively and electrical energy is generated, and the other is a light emitting device to generate photoenergy from electrical energy by supplying a voltage or a current to electrodes.

Examples of the organic optoelectronic device may include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode converts electrical energy into light, and the performance of organic light emitting diode is greatly influenced by the organic materials disposed between electrodes.

### SUMMARY

The embodiments may be realized by providing a compound for an organic optoelectronic device, the compound being represented by Chemical Formula 1:

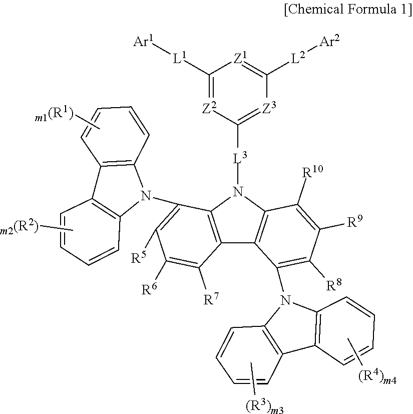

wherein, in Chemical Formula 1, Z¹ to Z³ are each independently N or CR^{a}, at least two of Z¹ to Z³ being N, L¹ and L² are each independently a single bond, a substituted or unsubstituted C6 to C30 arylene group, or a substituted or unsubstituted C2 to C30 heterocyclic group, L³ is a single bond or a substituted or unsubstituted C6 to C30 arylene group, Ar¹ and Ar² are each independently a substituted or unsubstituted C6 to C30 aryl group or a substituted or unsubstituted C2 to C30 heterocyclic group, R^{a} and R¹ to R¹⁰ are each independently hydrogen, deuterium, a substituted or unsubstituted C1 to C20 alkyl group, or a substituted or unsubstituted C6 to C20 aryl group, and m1 to m4 are each independently an integer of 1 to 4.

The embodiments may be realized by providing an organic optoelectronic device including an anode and a cathode facing each other, and at least one organic layer between the anode and the cathode, wherein the at least one organic layer includes a light emitting layer, and the light emitting layer includes the compound according to an embodiment.

The embodiments may be realized by providing a display device comprising the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view illustrating an organic light emitting diode according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples, “substituted” refers to replacement of at least one hydrogen of a substituent or a compound by deuterium, a C1 to C20 alkyl group, a C6 to C30 aryl group, or a cyano group. In addition, in specific examples, “substituted” refers to replacement of at least one hydrogen of a substituent or a compound by deuterium, a C1 to C5 alkyl group, a C6 to C18 aryl group, or a cyano group. In addition, in specific examples, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group a substituted or unsubstituted dibenzosilolyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a compound for an organic optoelectronic device according to an embodiment is described.

The compound for an organic optoelectronic device according to an embodiment may be represented by, e.g., Chemical Formula 1.

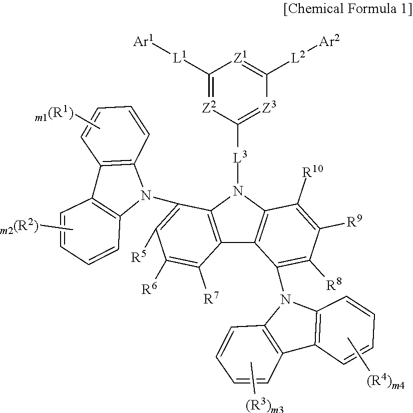

In Chemical Formula 1, Z¹ to Z³ may each independently be, e.g., N or CR^{a}. In an implementation, at least two of Z¹ to Z³ may be N.

L¹ and L² may each independently be or include, e.g., a single bond, a substituted or unsubstituted C6 to C30 arylene group, or a substituted or unsubstituted C2 to C30 heterocyclic group.

L³ may be or may include, e.g., a single bond or a substituted or unsubstituted C6 to C30 arylene group.

Ar¹ and Ar² may each independently be or include, e.g., a substituted or unsubstituted C6 to C30 aryl group or a substituted or unsubstituted C2 to C30 heterocyclic group.

R^{a} and R¹ to R¹⁰ may each independently be or include, e.g., hydrogen, deuterium, a substituted or unsubstituted C1 to C20 alkyl group, or a substituted or unsubstituted C6 to C20 aryl group.

m1 to m4 may each independently be, e.g., an integer of 1 to 4.

The compound represented by Chemical Formula 1 may have a structure in which carbazole is substituted with triazine at the 9-position (e.g., N-position), and the carbazole substituted with triazine at the 9-position may be further substituted with two 9-carbazoles (N-carbazole) at the 1-position and 5-position, respectively.

The compound may be substituted with the two 9-carbazoles at the 1-position and 5-position, respectively, and halving the π-bonding, which is a movement path of electrons and holes, may be addressed, thereby improving mobility of electrons and holes. In addition, the compound may have a plate-shaped molecular structure having a distorted structure due to steric hindrance by adjacent hydrogens or substituents, thereby realizing a low deposition temperature.

The organic light emitting diode to which the above material is applied may realize low driving voltage and high lifespan characteristics.

In addition, a material with a reduced T1 and S1 difference (ΔEst) may be prepared through a rigid structure design, and an organic light emitting diode to which such a material is applied may realize high efficiency.

In an implementation, L³ may be, e.g., a single bond or a substituted or unsubstituted phenylene group.

In an implementation, L³ may be, e.g., a substituted or unsubstituted ortho-phenylene group.

In an implementation, L³ may be a substituted or unsubstituted ortho-phenylene group, and it may have a steric hindrance compared to para-phenylene, thereby realizing a low deposition temperature. A carbazole group having HT properties and a triazine group having ET properties could be linked with para-phenylene, and localization (electron cloud localization) may occur only by the C—N bond that breaks the π-bond. When they are linked with ortho-phenylene, structurally, the group having HT properties and the group having ET properties are bonded at an angle to prevent electron movement and thus the HOMO/LUMO electron cloud localization is made clearer, a difference between T1 and S1 may be reduced, a material with a small ΔEst may be realized. Materials with a small ΔEst may typically achieve high efficiencies.

In an implementation, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzosilolyl group, a substituted or unsubstituted dibenzofuranyl group, a substituted or unsubstituted dibenzothiophenyl group, or a substituted or unsubstituted carbazolyl group.

In an implementation, Ar¹ and Ar² may each independently be, e.g., a substituted or unsubstituted phenyl group, a substituted or unsubstituted biphenyl group, a substituted or unsubstituted naphthyl group, a substituted or unsubstituted dibenzosilolyl group, a substituted or unsubstituted dibenzofuranyl group, or a substituted or unsubstituted dibenzothiophenyl group.

In an implementation, L¹ and L² may each independently be, e.g., a single bond, a substituted or unsubstituted phenylene group, or a substituted or unsubstituted naphthylene group.

In an implementation, moieties *-L¹-Ar¹ and *-L²-Ar² may each independently be, e.g., a moiety of Group I.

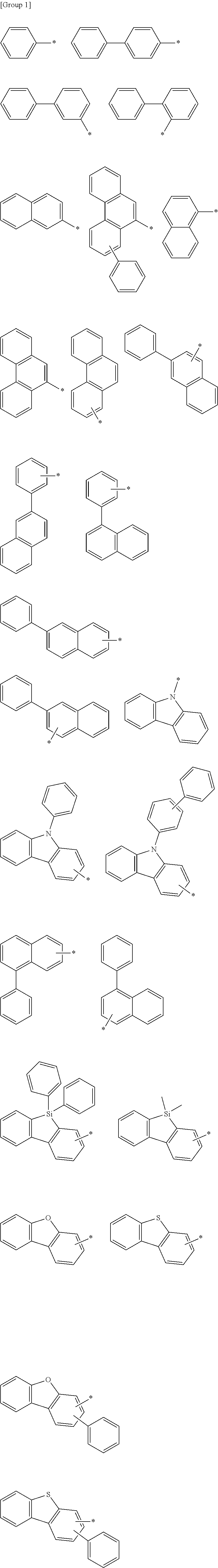

In Group I, * is a linking point,

In an implementation, the moieties of Group I may be unsubstituted (e.g., as illustrated) or may be substituted with a suitable substituent.

In an implementation, the substituent may be, e.g., deuterium, a C1 to C5 alkyl group, a C6 to C18 aryl group, or a cyano group.

In an implementation, the compound for the organic optoelectronic device represented by Chemical Formula 1 may be, e.g., a compound of Group 1.

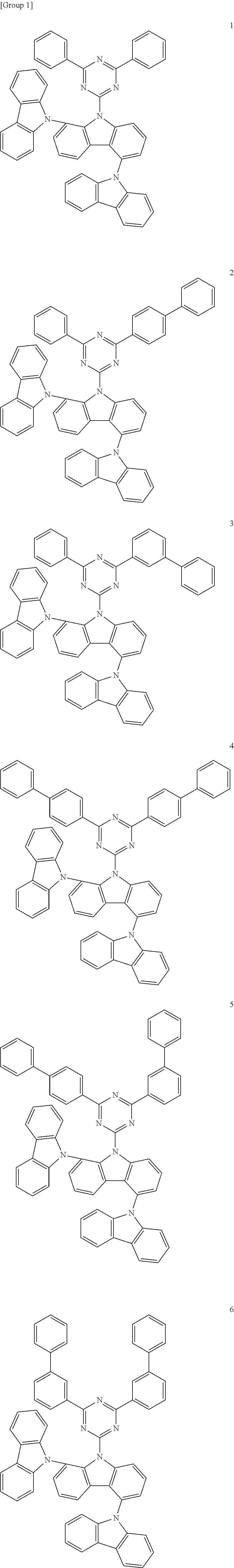

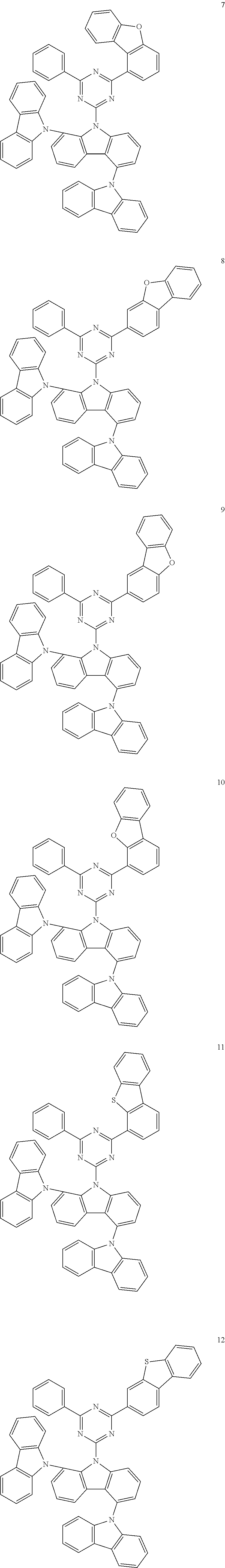

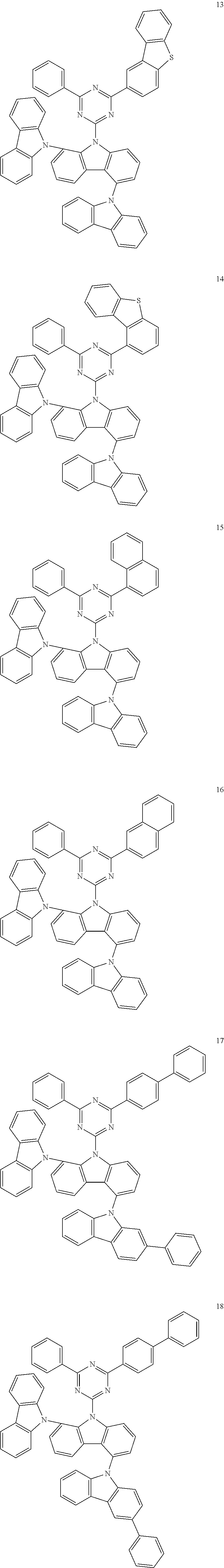

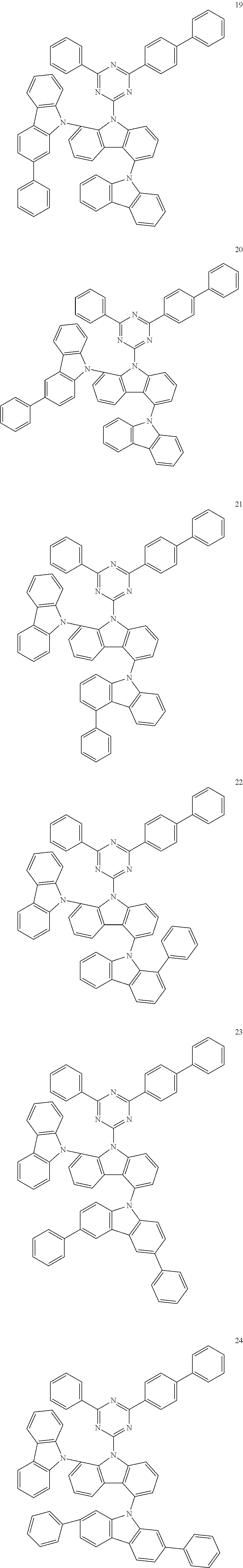

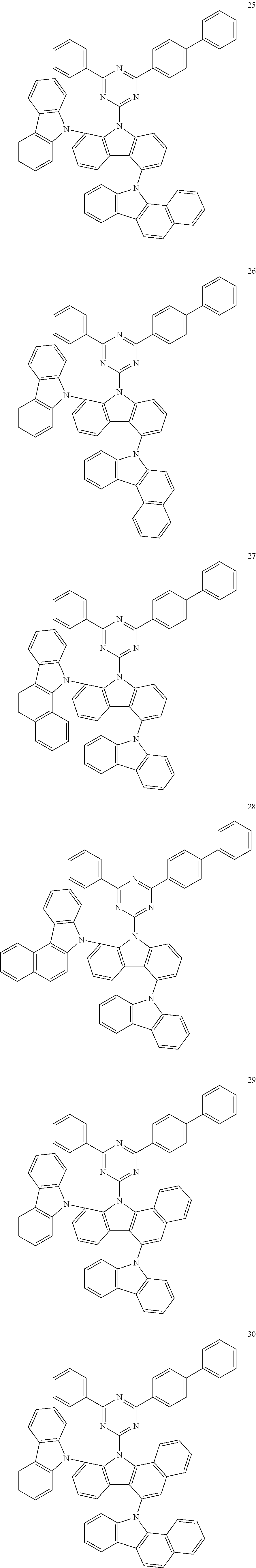

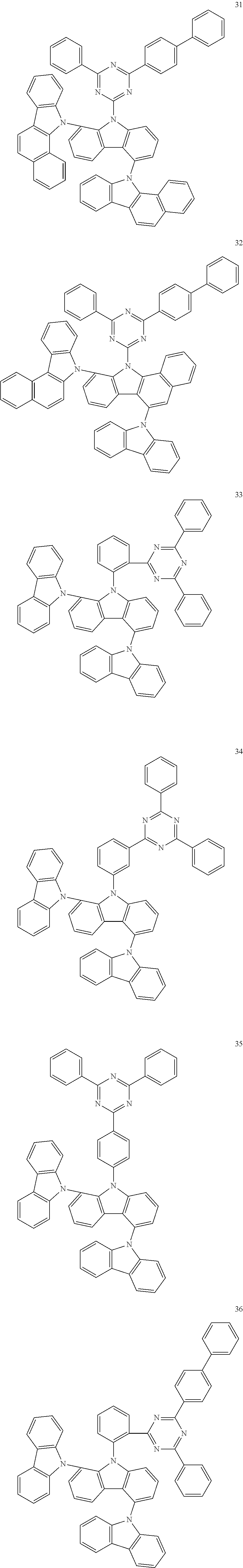

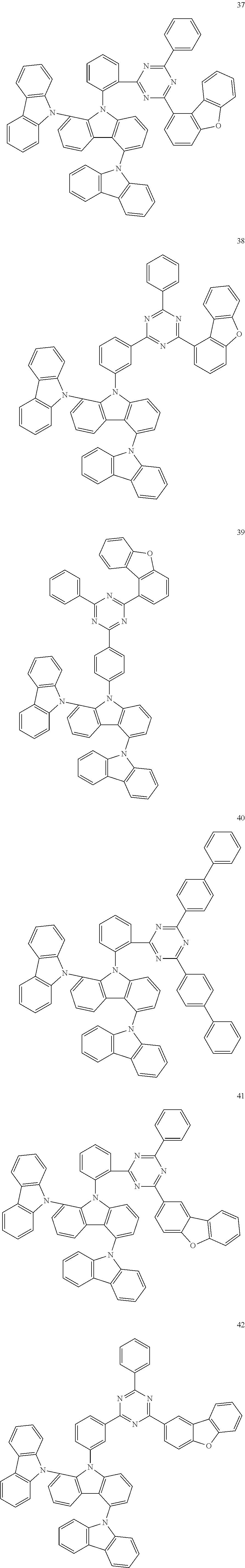

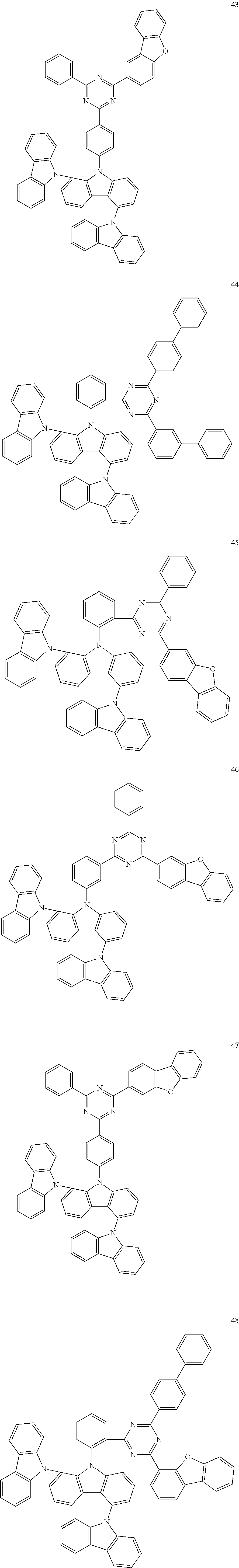

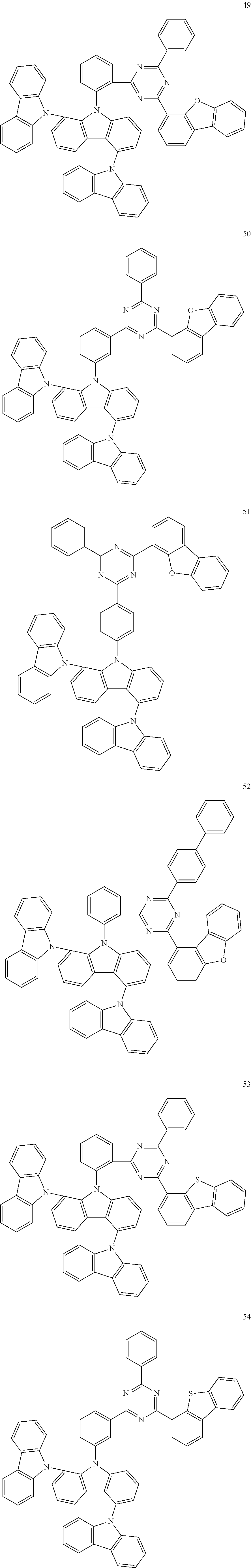

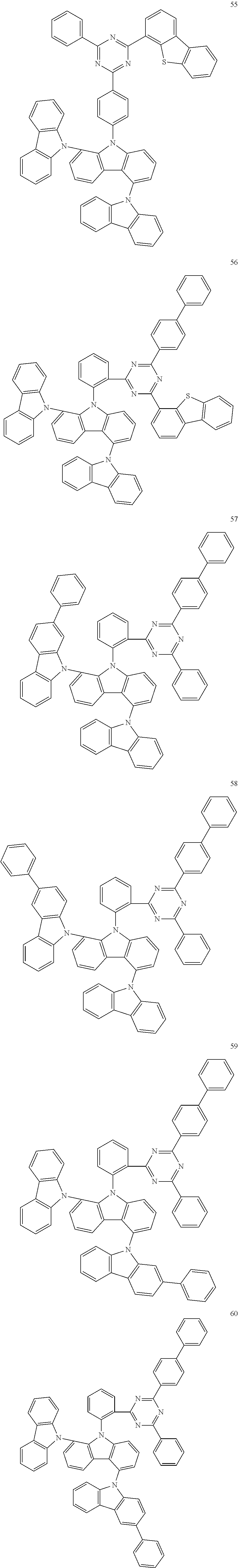

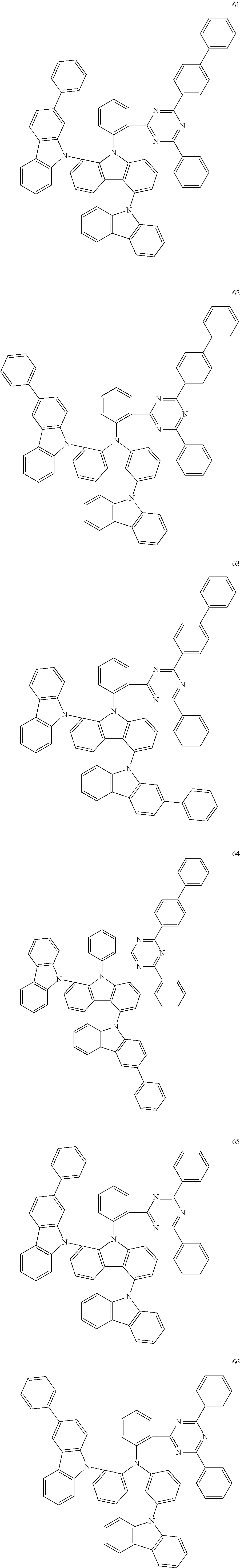

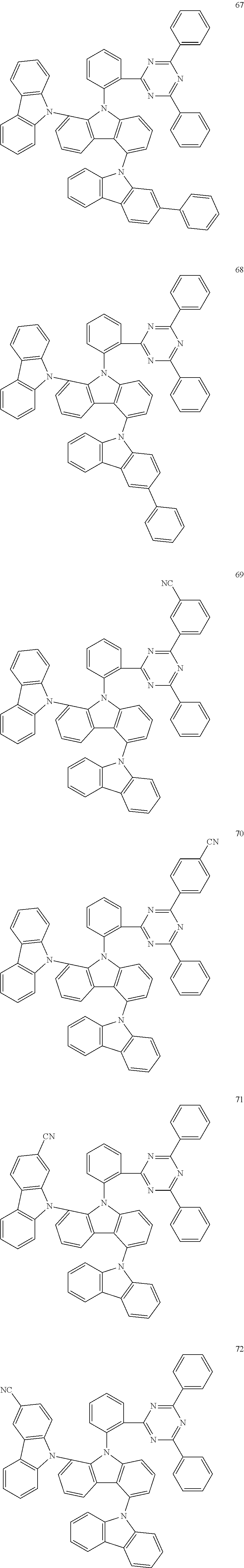

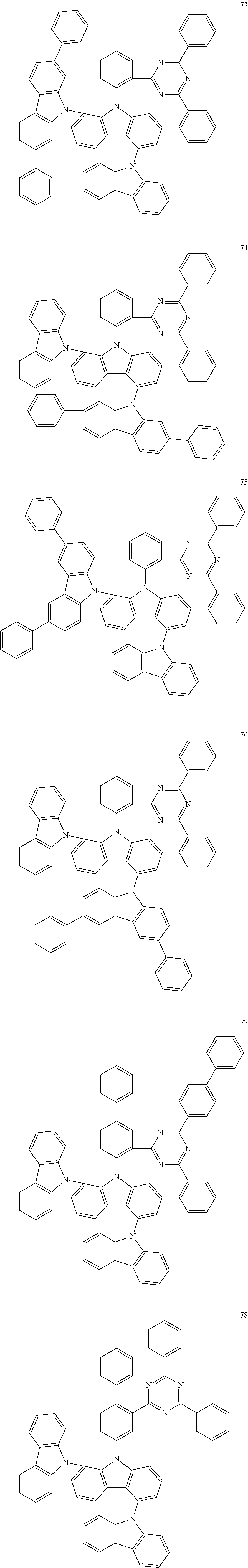

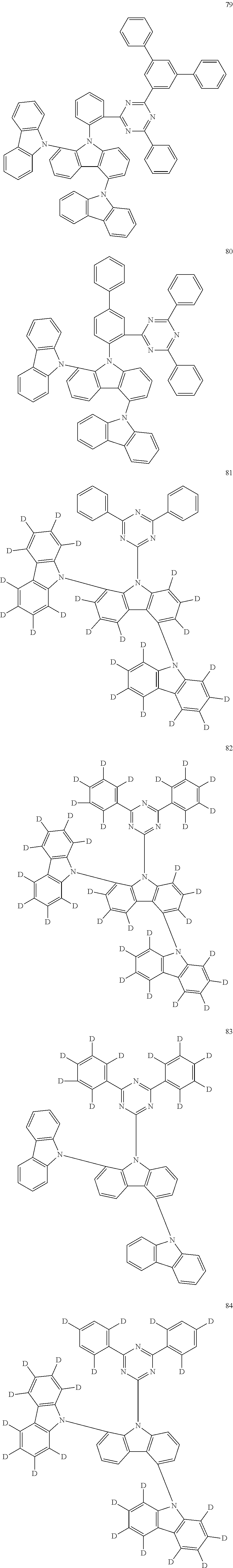

wherein D is deuterium.

The aforementioned compound for an organic optoelectronic device may be used, e.g., as a host. In an implementation, the compound may be mixed with a suitable host material.

The aforementioned compound for an organic optoelectronic device may be included in a composition that further includes a dopant.

The dopant may be, e.g., a phosphorescent dopant, for example a red, green or blue phosphorescent dopant, for example a red or green phosphorescent dopant.

The dopant is a material mixed with the compound or composition for the organic optoelectronic device in a small amount to cause light emission and may be a material such as a metal complex that emits light by multiple excitation into a triplet or more. The dopant may be, e.g., an inorganic, organic, or organic-inorganic compound, and one or more types thereof may be used.

Examples of the dopant may include a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, e.g., a compound represented by Chemical Formula Z.

L⁴MX [Chemical Formula Z]

In Chemical Formula Z, M may be a metal, and L⁴ and X are the same as or different from each other, and may be ligands forming a complex compound with M.

The M may be, e.g., Ir, Pt, Os, Ti, Zr, Hf, Eu, Tb, Tm, Fe, Co, Ni, Ru, Rh, Pd, or a combination thereof, and L⁴ and X may be, e.g., a bidentate ligand.

The ligands represented by L⁴ and X may be, e.g., represented by the chemical formulae of Group A.

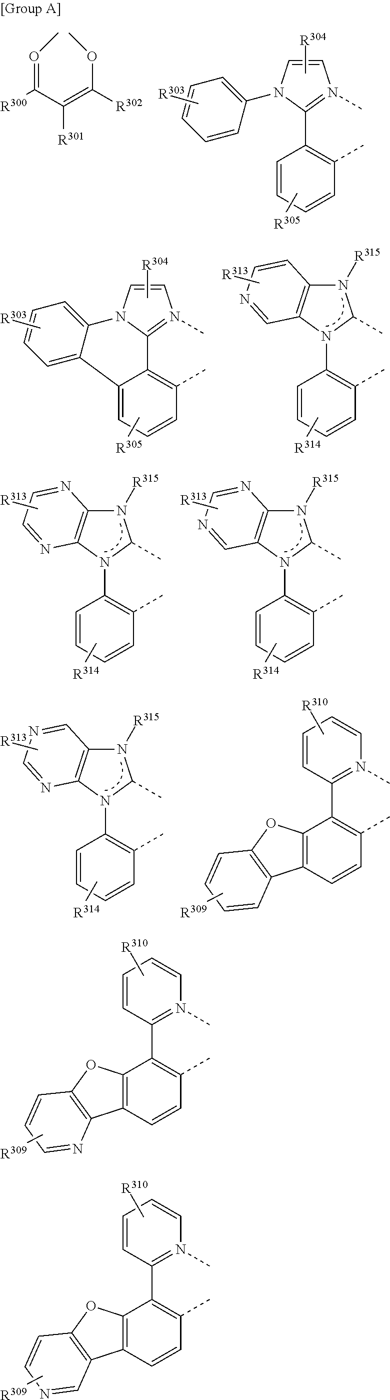

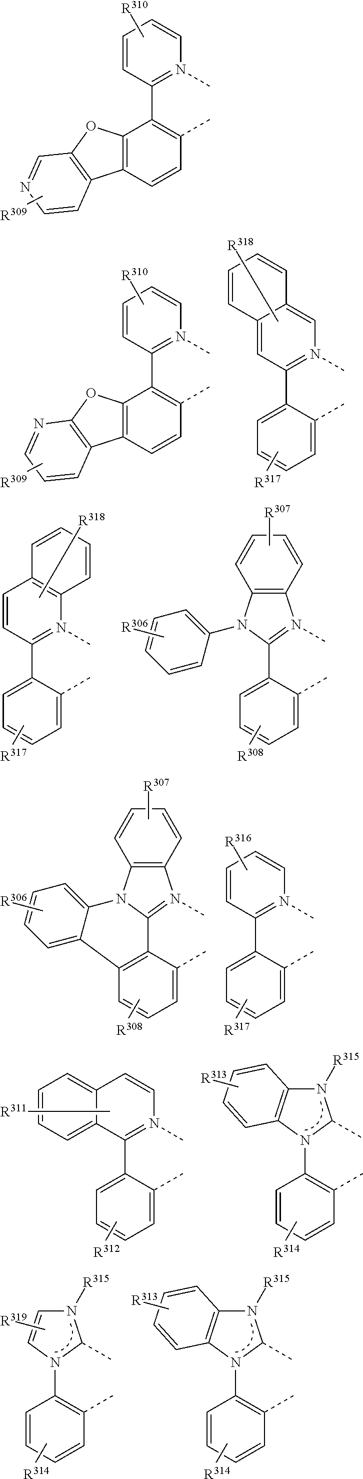

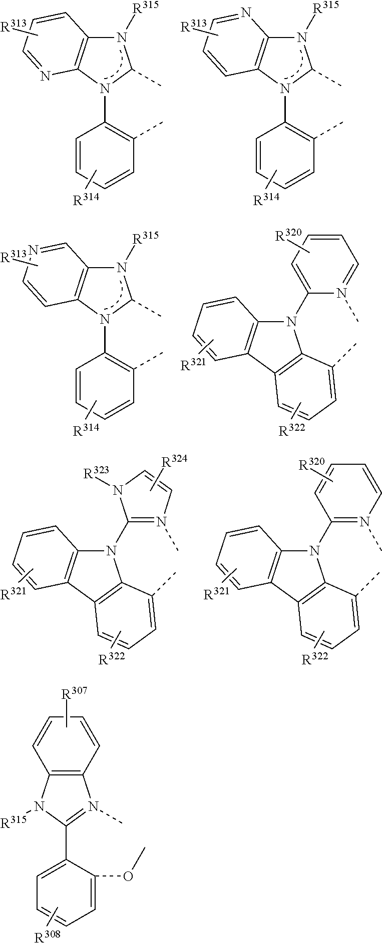

In Group A, R³⁰⁰ to R³⁰² may each independently be hydrogen, deuterium, a C1 to C30 alkyl group that is substituted or unsubstituted with a halogen, a C6 to C30 aryl group that is substituted or unsubstituted with a C1 to C30 alkyl, or a halogen, and

R³⁰³ to R³²⁴ may each independently be hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

In an implementation, it may include a dopant represented by Chemical Formula I.

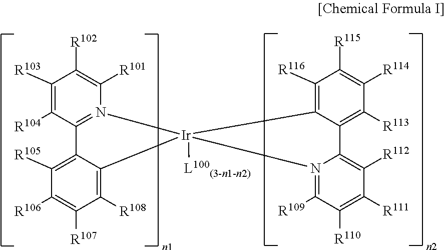

In Chemical Formula I, R¹⁰¹ to R¹¹⁶ may each independently be hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ may each independently be a C1 to C6 alkyl group,

at least one of R¹⁰¹ to R¹¹⁶ may be a functional group represented by Chemical Formula I-1,

L¹⁰⁰ may be a bidentate ligand of a monovalent anion, and is a ligand that coordinates to iridium through a lone pair of electrons of carbon or heteroatom, and

n1 and n2 may each independently be an integer of 0 to 3, and n1+n2 may be an integer of 1 to 3.

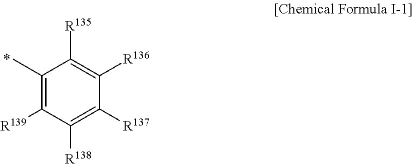

In Chemical Formula I-1, R¹³⁵ to R¹³⁹ may each independently be hydrogen, deuterium, a substituted or unsubstituted C1 to C10 alkyl group, a substituted or unsubstituted C6 to C20 aryl group, or —SiR¹³²R¹³³R¹³⁴, and

* means a portion linked to a carbon atom.

In an implementation, a dopant represented by Chemical Formula Z-1 may be included.

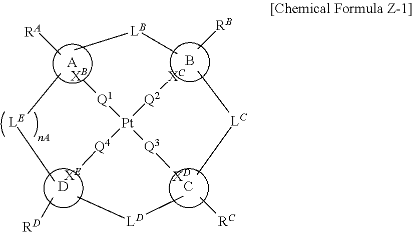

In Chemical Formula Z-1, rings A, B, C, and D may each independently be a 5-membered or 6-membered carbocyclic or heterocyclic ring;

R^{A}, R^{B}, R^{C}, and R^{D} may each independently be mono-, di-, tri-, or tetra-substitution, or an un-substitution;

L^{B}, L^{C}, and L^{D} may each independently be a direct bond, BR, NR, PR, O, S, Se, C═O, S═O, SO₂, CRR′, SiRR′, GeRR′, and a combination thereof,

when nA is 1, L^{E} is a direct bond, BR, NR, PR, O, S, Se, C═O, S═O, SO₂, CRR′, SiRR′, GeRR′, or a combination thereof, when nA is 0, L^{E} does not exist; and

R^{A}, R^{B}, R^{C}, R^{D}, R, and R′ may each independently be hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ may be optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be, e.g., represented by Chemical Formula II.

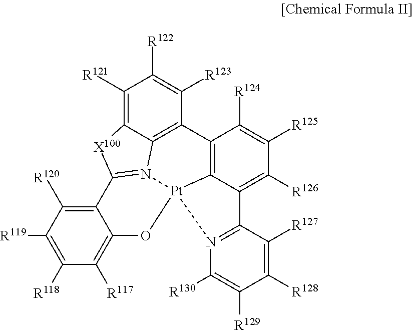

In Chemical Formula II, X¹⁰⁰ may be O, S, or NR¹³¹,

R¹¹⁷ to R¹³¹ may each independently be hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ may each independently be a C1 to C6 alkyl group, and

at least one of R¹¹⁷ to R¹³¹ may be —SiR¹³²R¹³³R¹³⁴ or a tert-butyl group.

Hereinafter, an organic optoelectronic device including the aforementioned compound for the organic optoelectronic device is described.

The organic optoelectronic device may be a suitable device to convert electrical energy into photoenergy and vice versa, e.g., an organic photoelectric device, an organic light emitting diode, an organic solar cell, or an organic photo-conductor drum.

Herein, an organic light emitting diode as one example of an organic optoelectronic device is described referring to drawings.

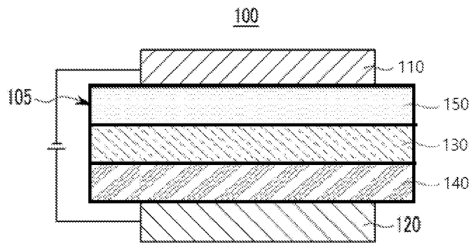

The FIG. 1s a cross-sectional view illustrating an organic light emitting diode according to an embodiment.

Referring to the FIGURE, an organic light emitting diode 100 according to an embodiment may include an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; or a multi-layer structure material such as LiF/Al, LiO₂/Al, LiF/Ca, or BaF₂/Ca.

The organic layer 105 may include the aforementioned compound for the organic optoelectronic device.

The organic layer 105 may include a light emitting layer 130 and the light emitting layer 130 may include the aforementioned compound for the organic optoelectronic device.

The composition for the organic optoelectronic device further including the dopant may be, e.g., a red or green light emitting composition.

The light emitting layer 130 may include, e.g., the aforementioned compound for the organic optoelectronic device as each phosphorescent host.

The organic layer may further include a charge transport region in addition to the light emitting layer.

The charge transport region may be, e.g., a hole transport region 140.

The hole transport region 140 may further increase hole injection or hole mobility between the anode 120 and the light emitting layer 130 and block electrons.

In an implementation, the hole transport region 140 may include a hole transport layer between the anode 120 and the light emitting layer 130, and a hole transport auxiliary layer between the light emitting layer 130 and the hole transport layer and at least one of the compounds of Group B may be included in at least one of the hole transport layer and the hole transport auxiliary layer.

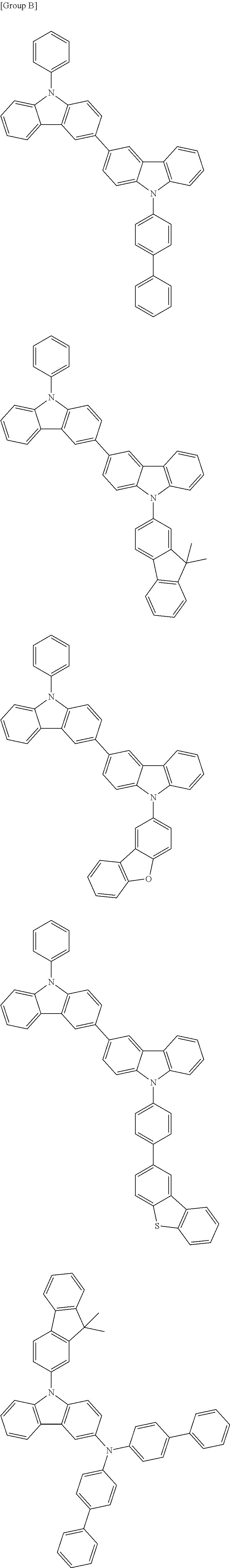 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

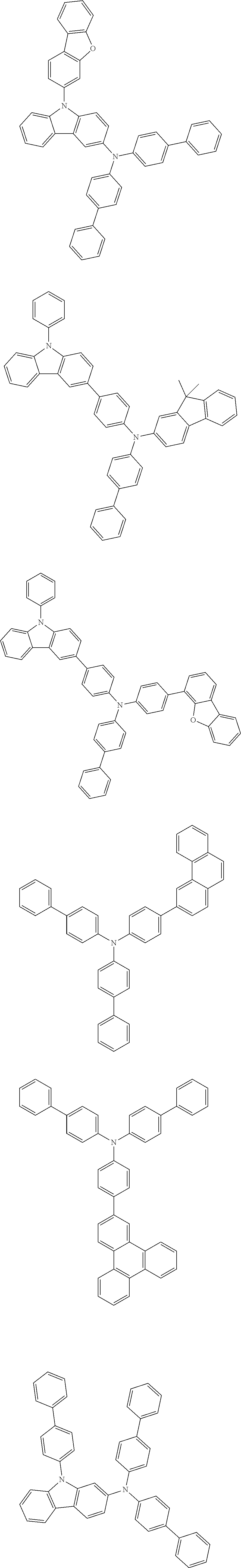

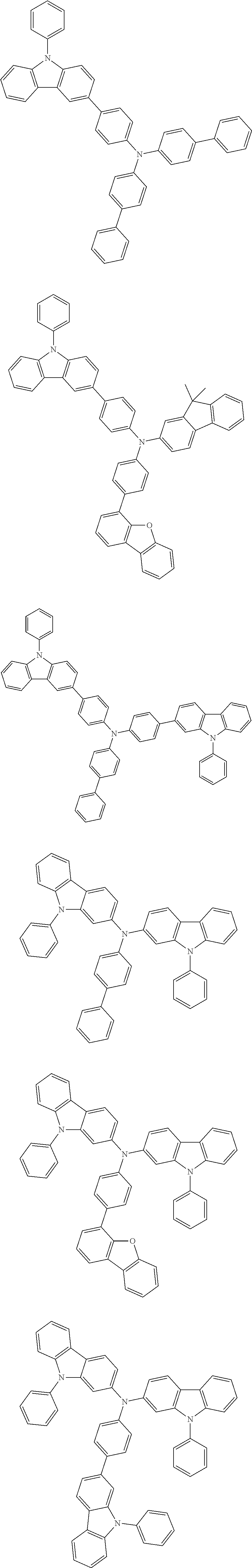

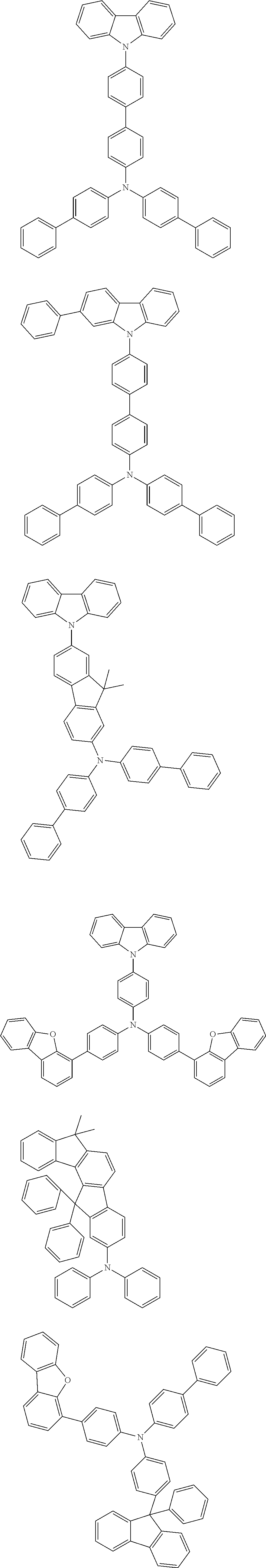

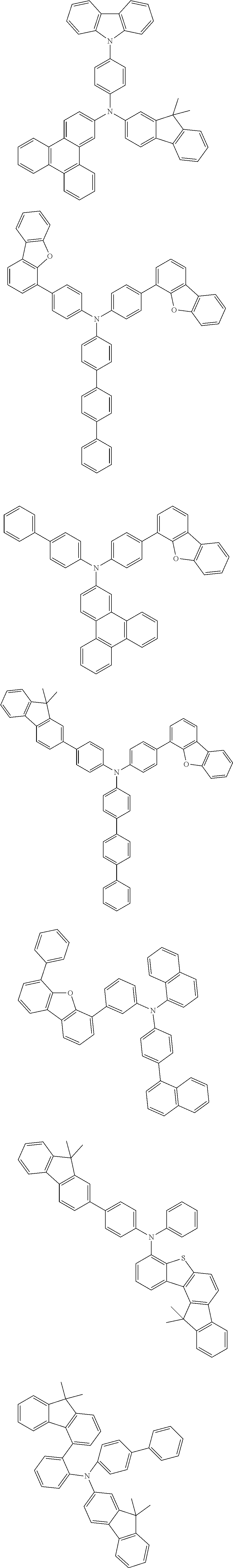

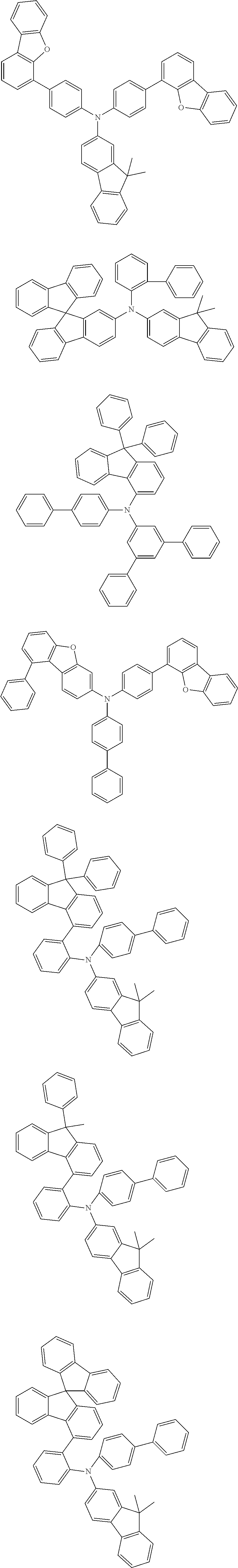

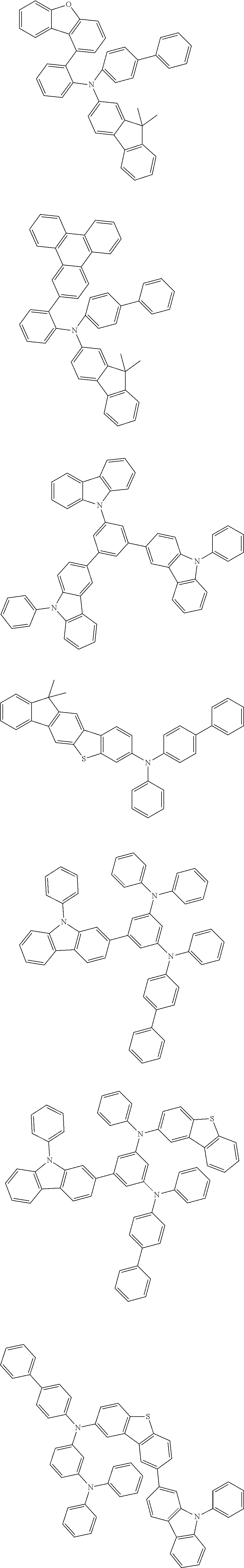

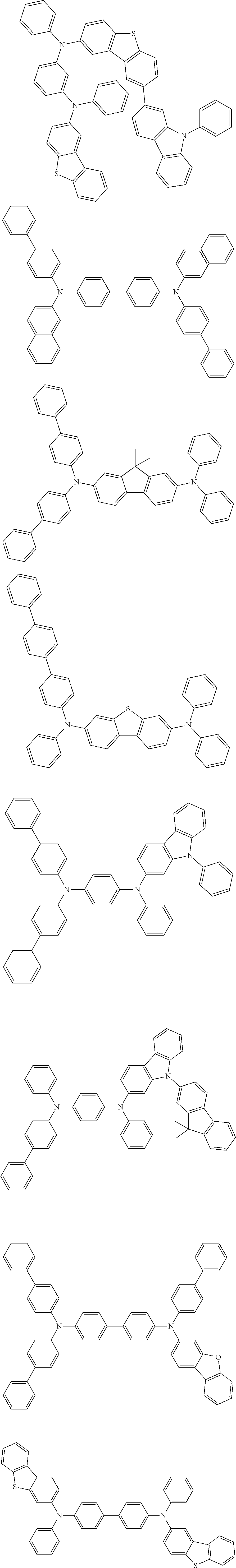

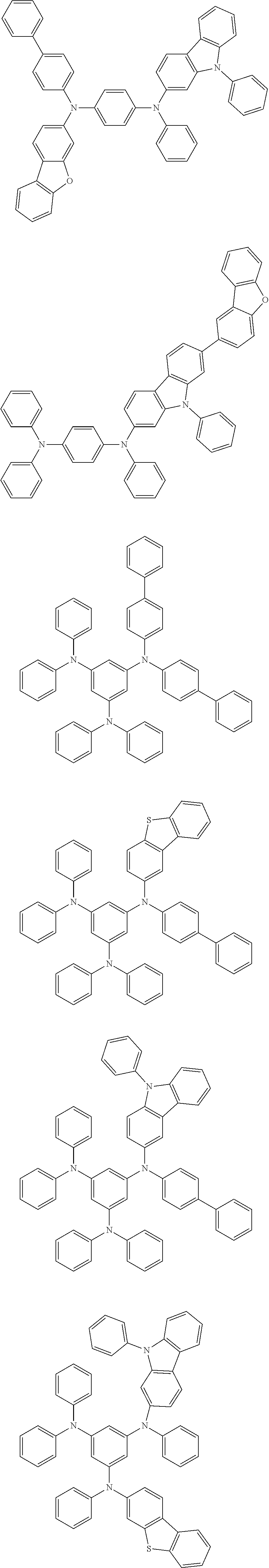

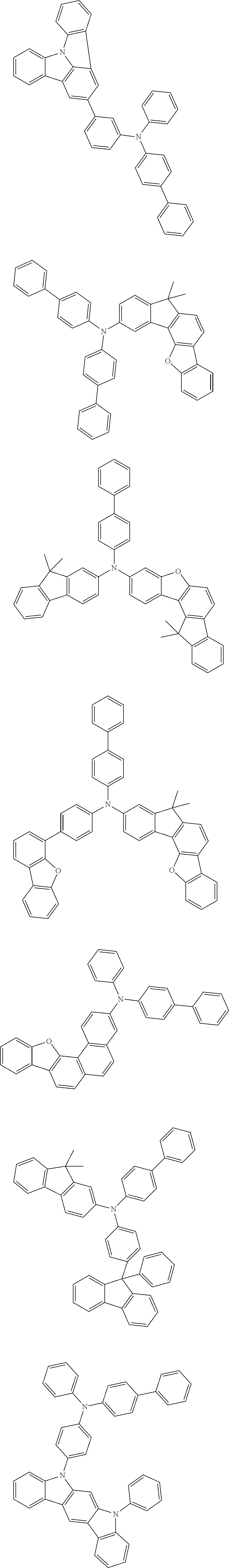

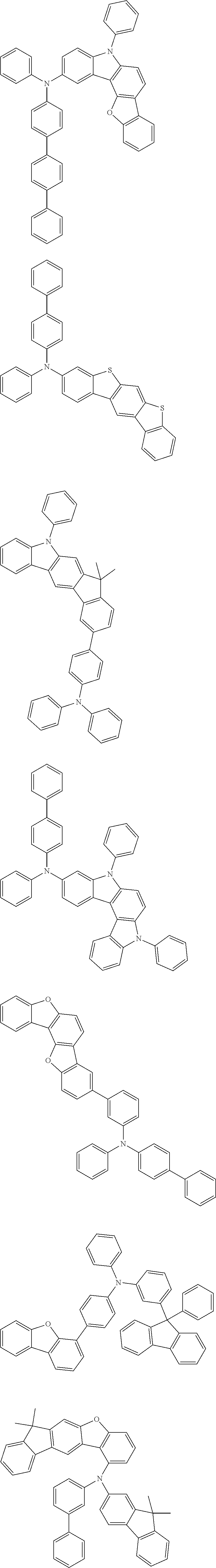

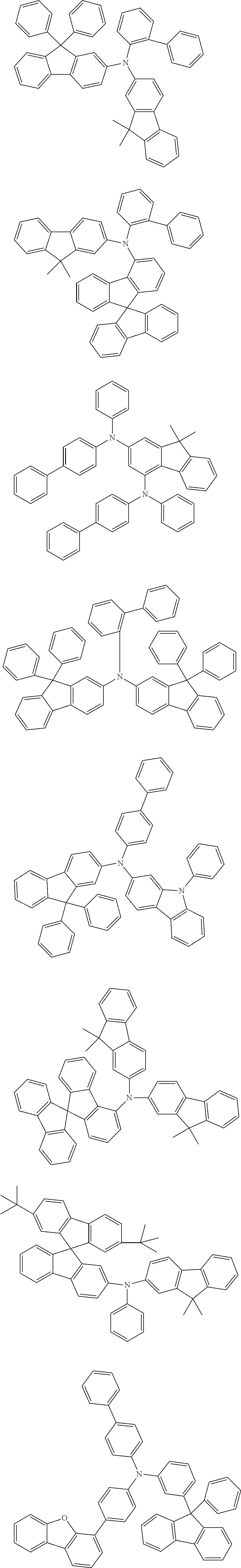

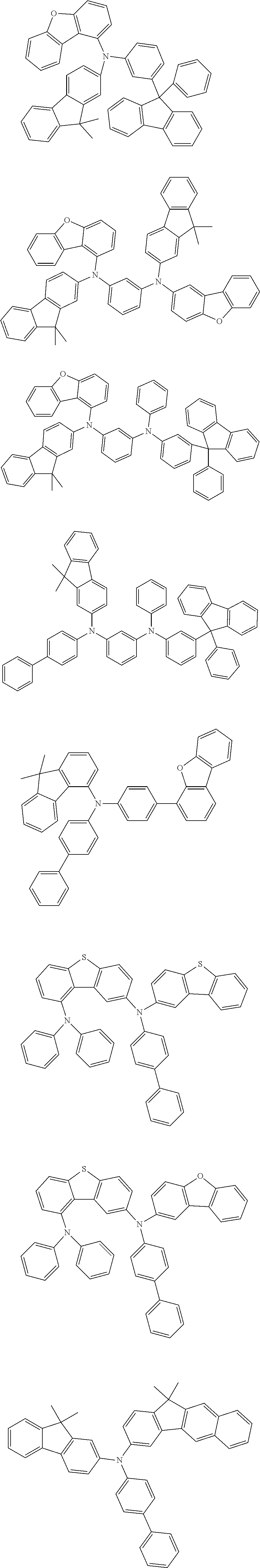

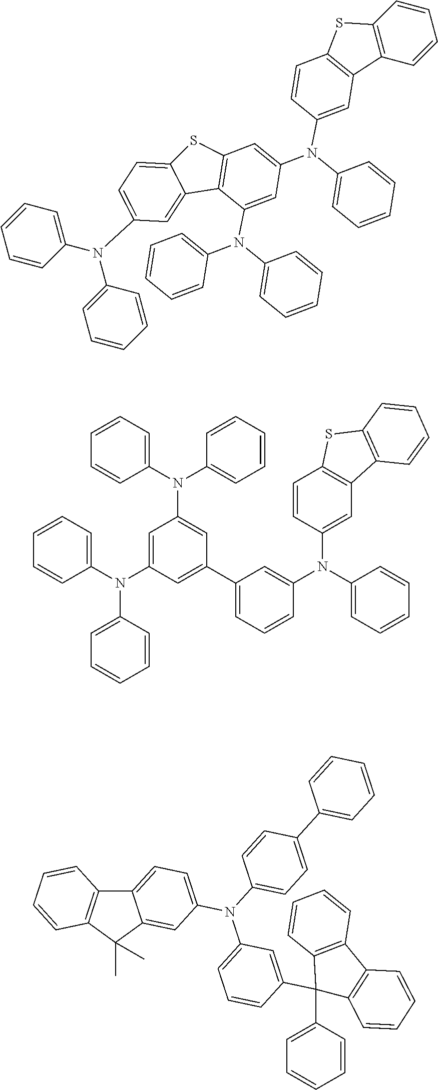 [SMILES: c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region 140, other suitable compounds may be used in addition to the compounds described above.

In an implementation, the charge transport region may be, e.g., an electron transport region 150.

The electron transport region 150 may further increase electron injection or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

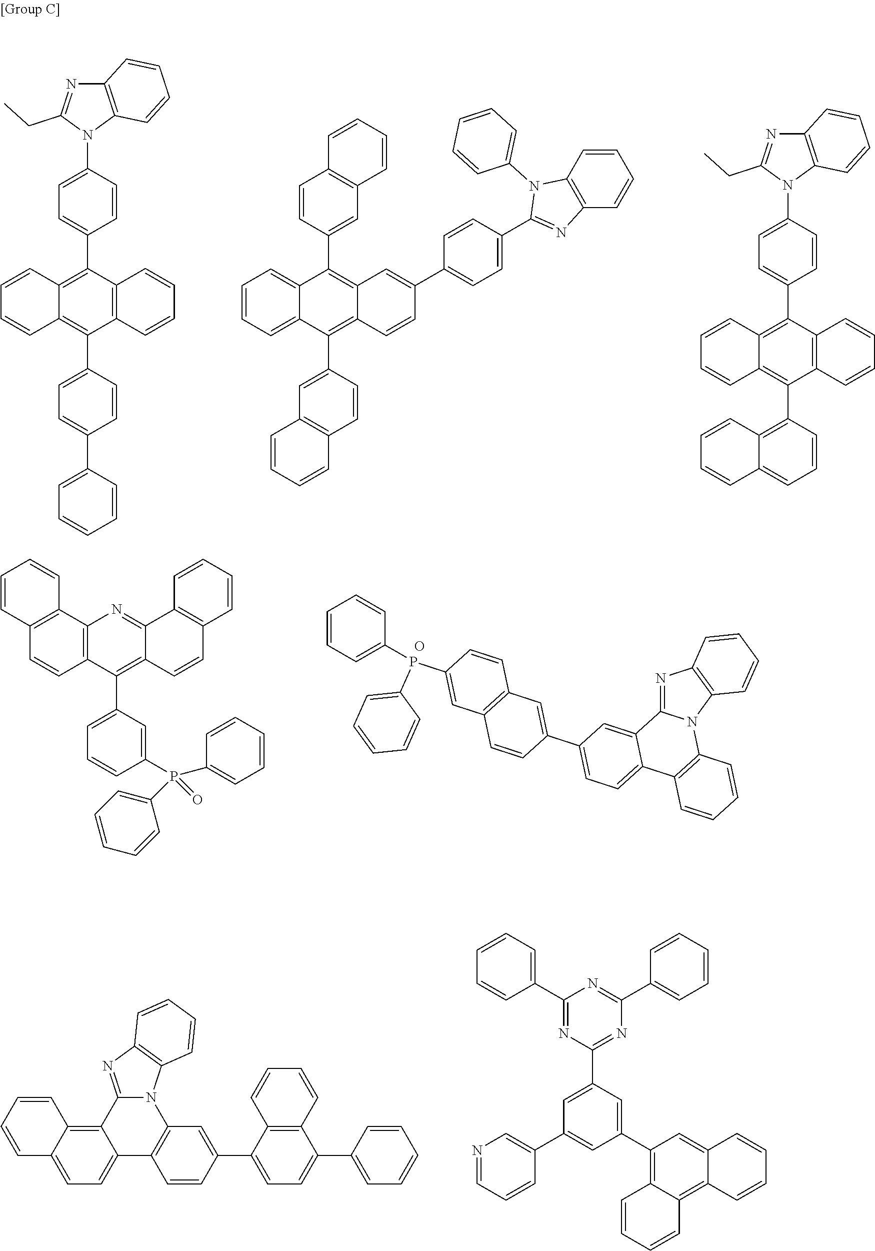

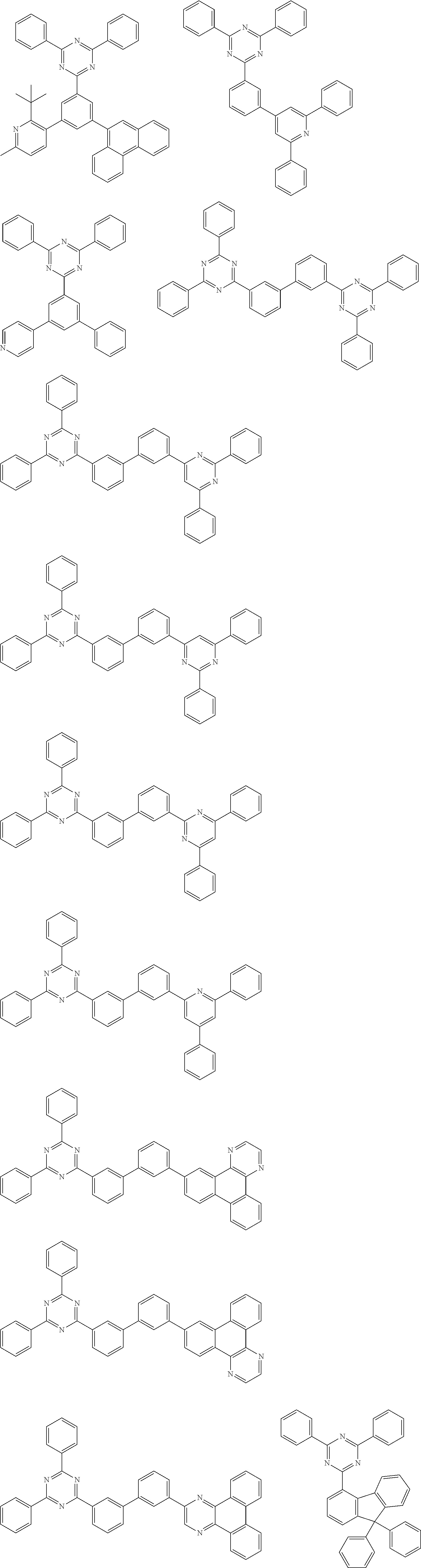

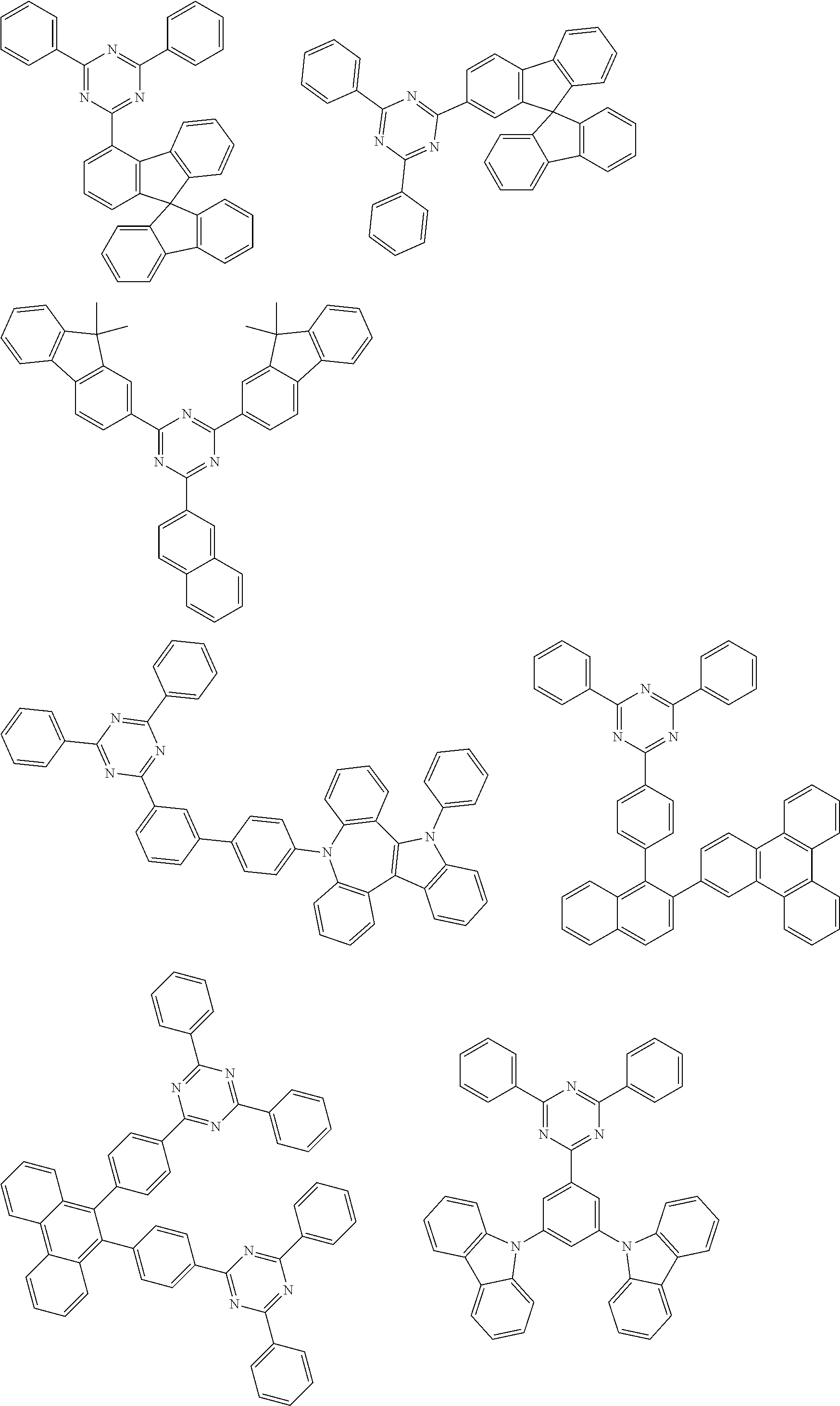

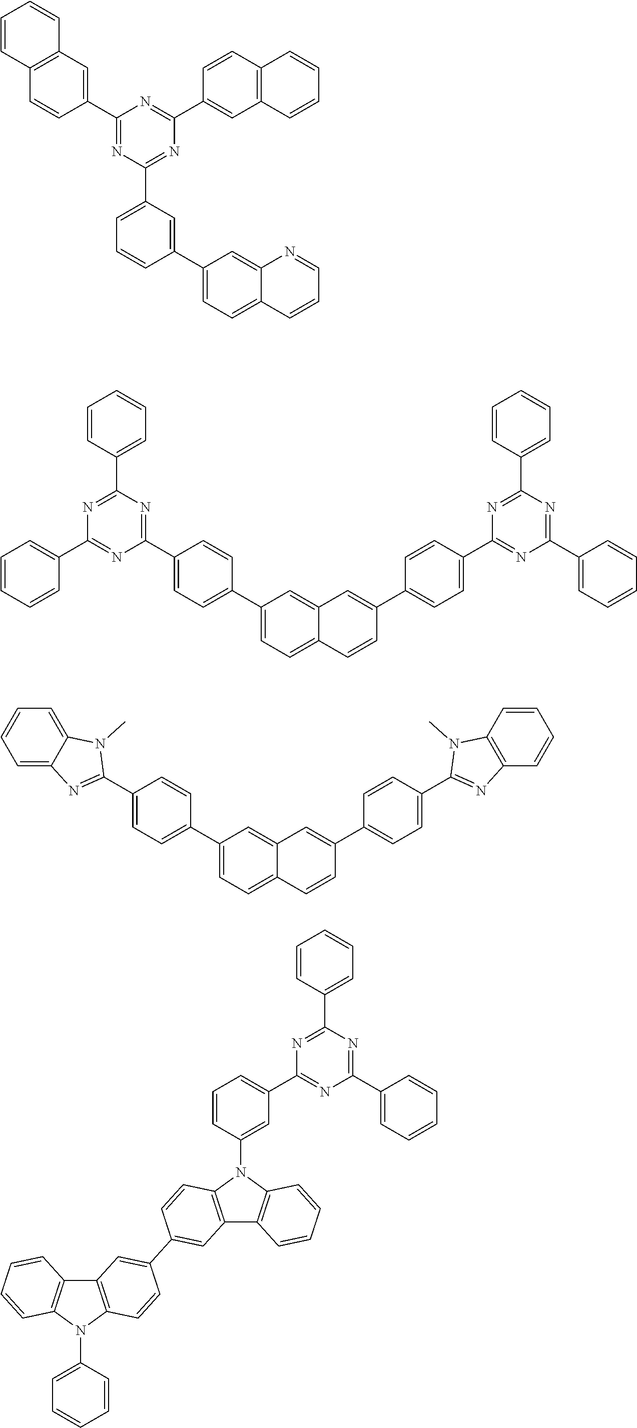

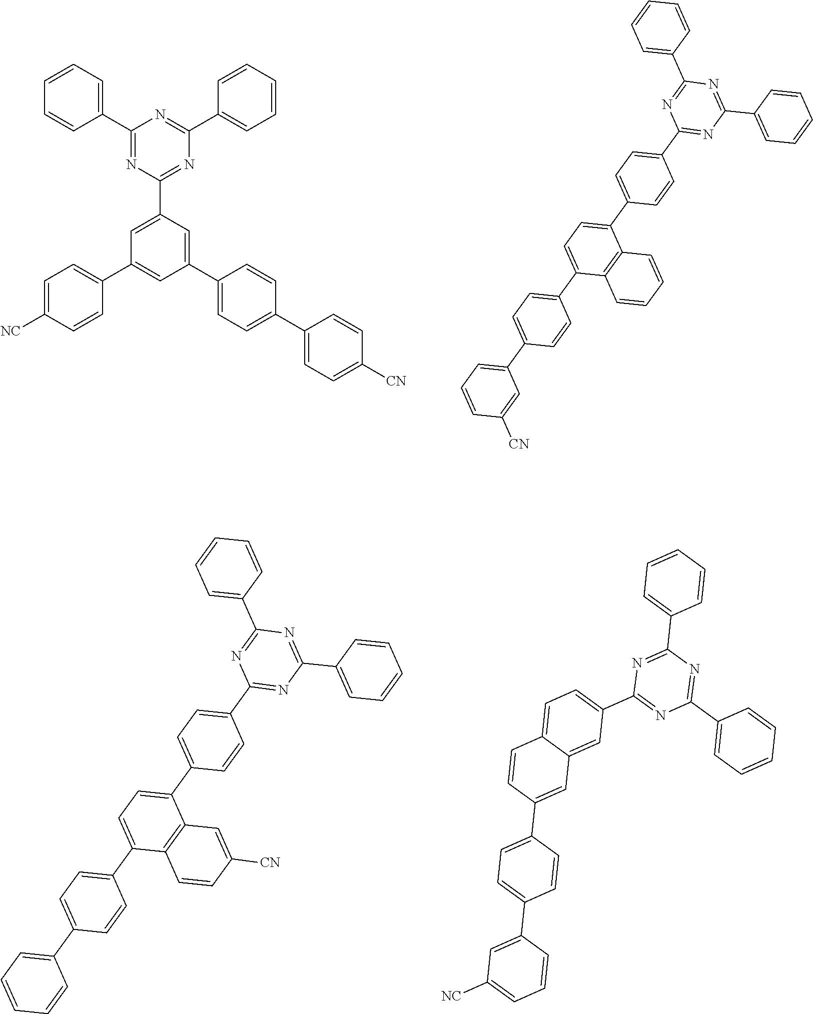 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1]

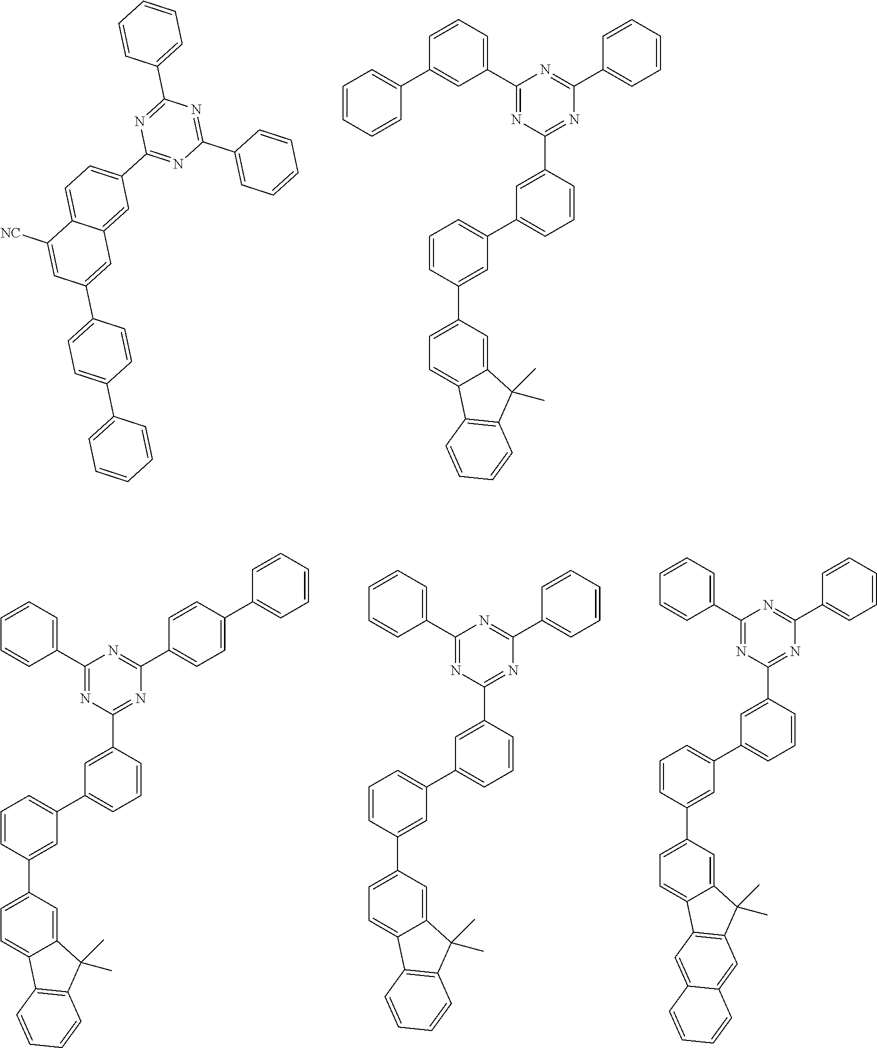 [SMILES: N#Cc1cc(-c2ccc(-c3ccccc3)cc2)cc2cc(-c3nc(-c4ccccc4)nc(-c4ccccc4)n3)ccc12 ; CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21 ; CC1(C)c2ccccc2-c2ccc(-c3cccc(-c4cccc(-c5nc(-c6ccccc6)nc(-c6ccccc6)n5)c4)c3)cc21 ; CC1(C)c2cc(-c3cccc(-c4cccc(-c5nc(-c6ccccc6)nc(-c6ccccc6)n5)c4)c3)ccc2-c2cc3ccccc3cc21]

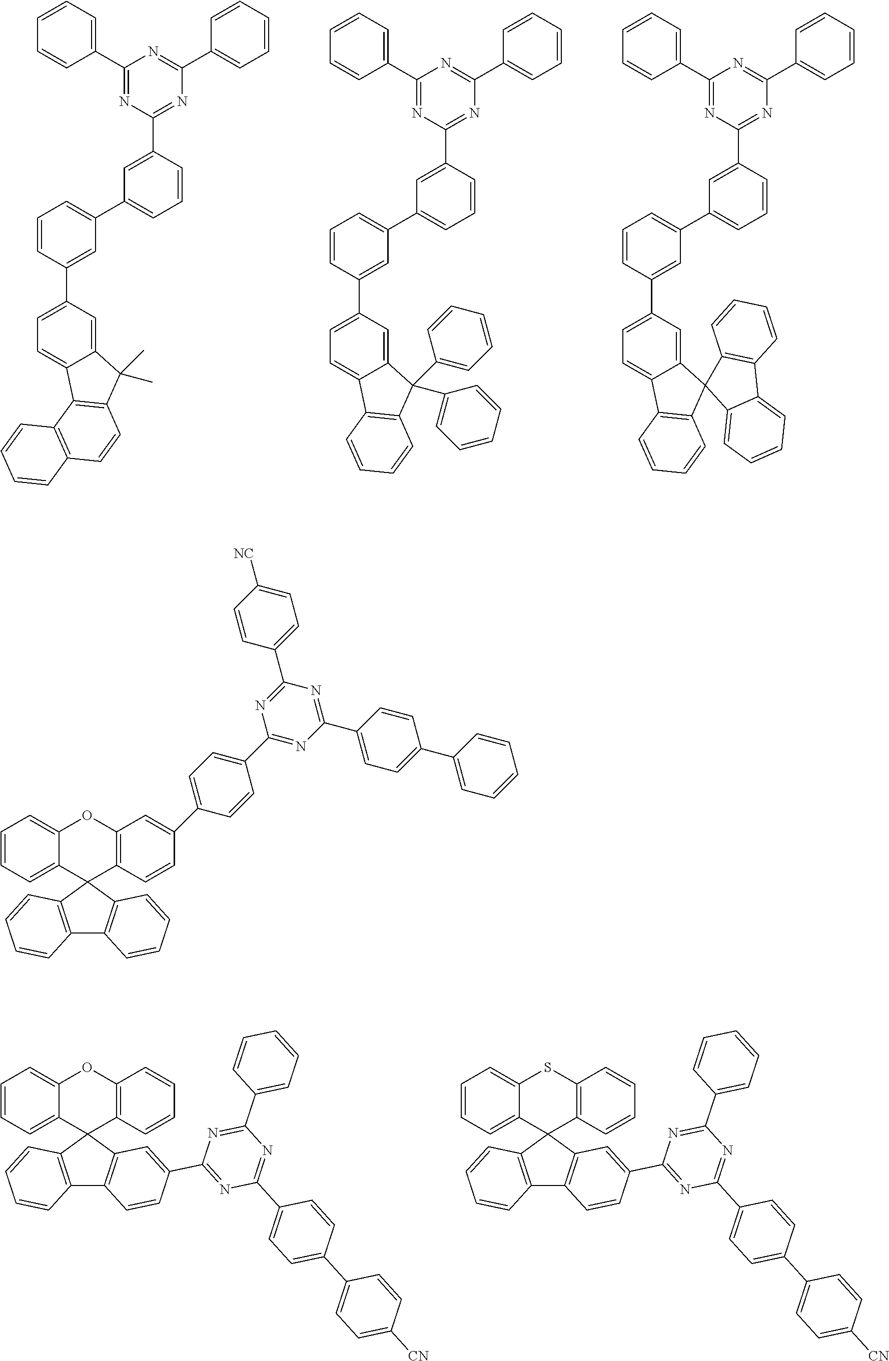

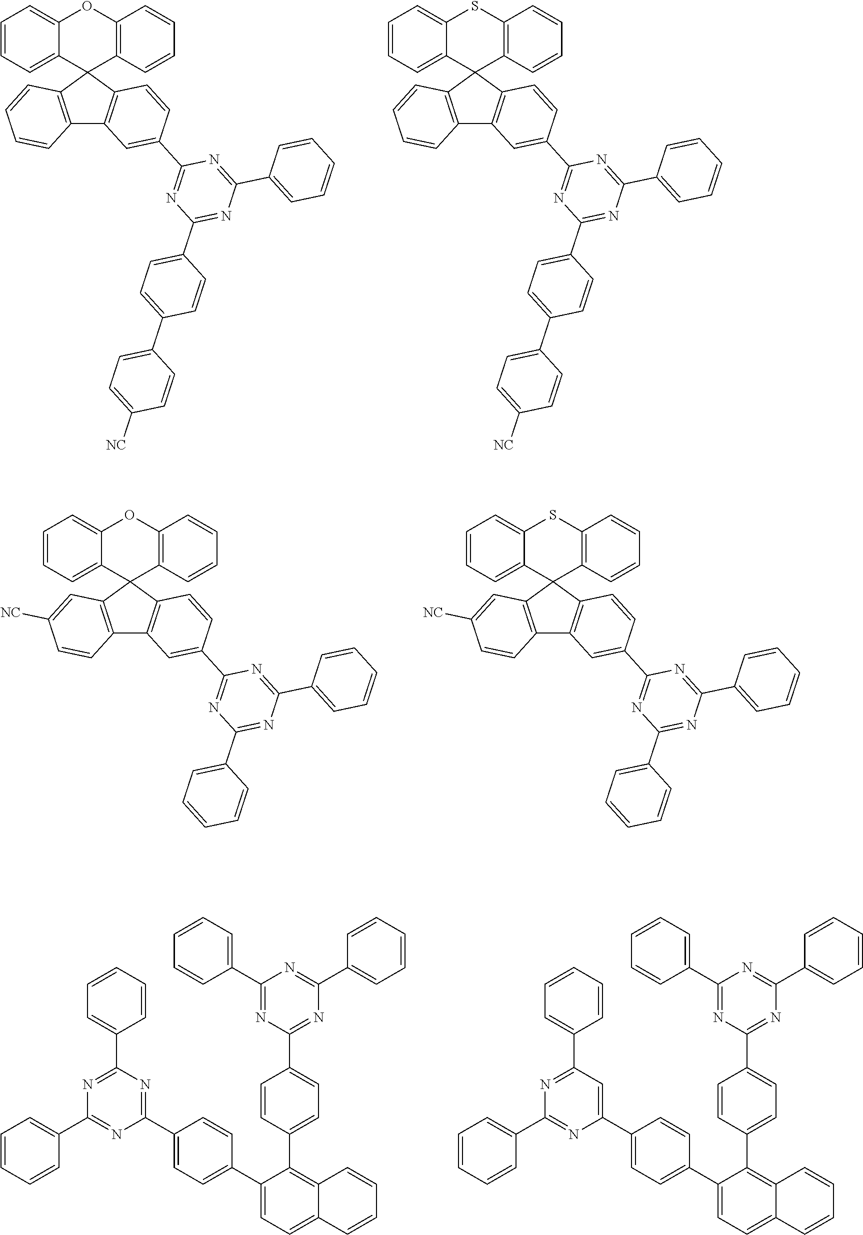

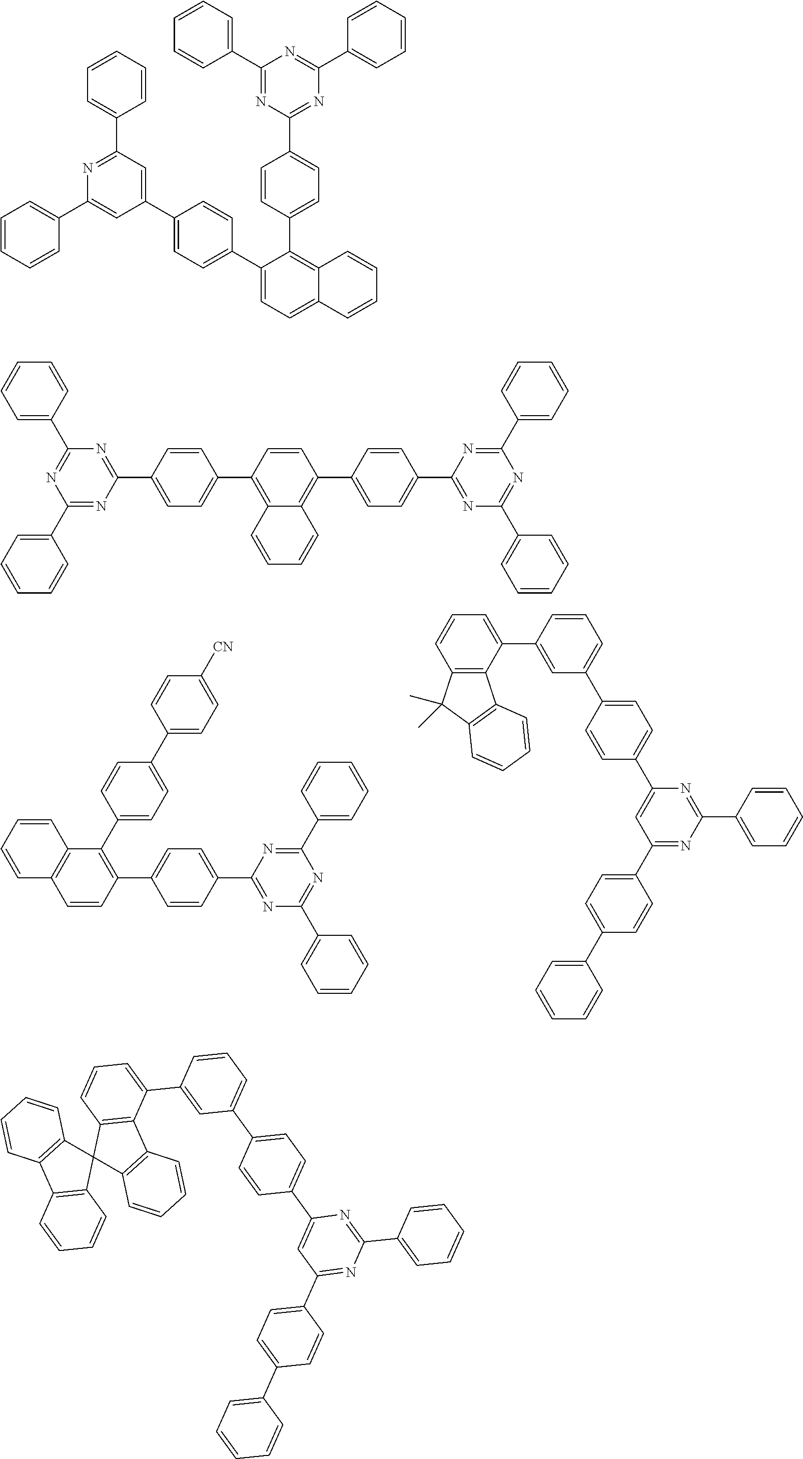

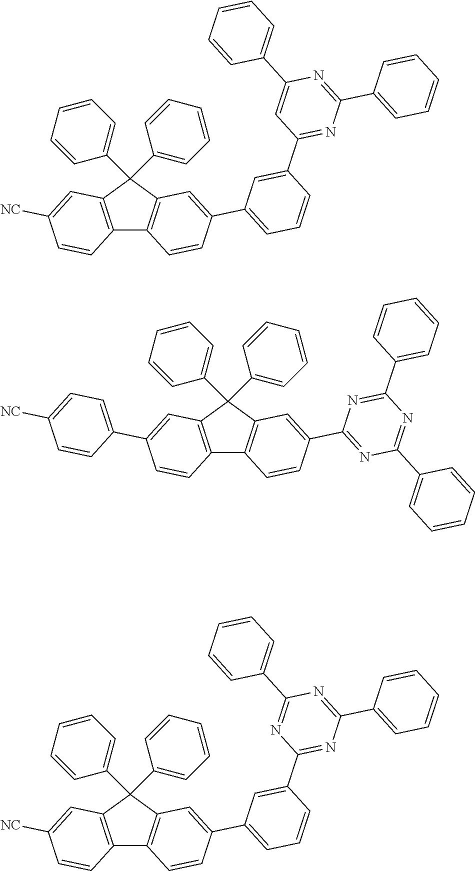 [SMILES: N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may be an organic light emitting diode including a light emitting layer as an organic layer.

Another embodiment may provide an organic light emitting diode including a light emitting layer and a hole transport region as an organic layer.

Another embodiment may provide an organic light emitting diode including a light emitting layer and an electron transport region as an organic layer.

As shown in the FIGURE, the organic light emitting diode according to the embodiment may include a hole transport region 140 and an electron transport region 150 in addition to the light emitting layer 130 as the organic layer 105.

In an implementation, the organic light emitting diode may further include an electron injection layer, a hole injection layer, or the like, in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., Tokyo Chemical Industry, or P&H Tech as far as there in no particular comment or were synthesized by suitable methods.

(Preparation of Compound for Organic Optoelectronic Device)

The compound was synthesized through the following steps.

### Synthesis Example 1: Synthesis of Intermediate I-1

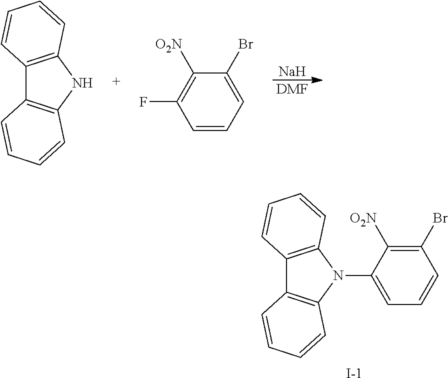

Under a nitrogen atmosphere, 9H-carbazole (100 g, 598 mmol) purchased from Tokyo Chemical Industry Co., Ltd. was dissolved in 0.1 L of dimethylformamide (DMF), and sodium hydride (28.7 g, 1,196 mmol) was added thereto and then, stirred at 0° C. After 1 hour, 1-bromo-3-fluoro-2-nitrobenzene (132 g, 598 mmol) purchased from Tokyo Chemical Industry Co., Ltd. was added thereto and then, stirred for 1 hour. When a reaction was completed, water was added to the reaction solution at 0° C. and then, extracted with dichloromethane (DCM), treated with anhydrous magnesium sulfate to remove moisture, filtered, and concentrated under a reduced pressure. The obtained residue was separated and purified through flash column chromatography, obtaining Intermediate I-1 (182 g, 83%).

HRMS (70 eV, EI+): m/z calcd for C18H11BrN2O2: 366.0004, found: 366.

Elemental Analysis: C, 59%; H, 3%

### Synthesis Example 2: Synthesis of Intermediate I-2

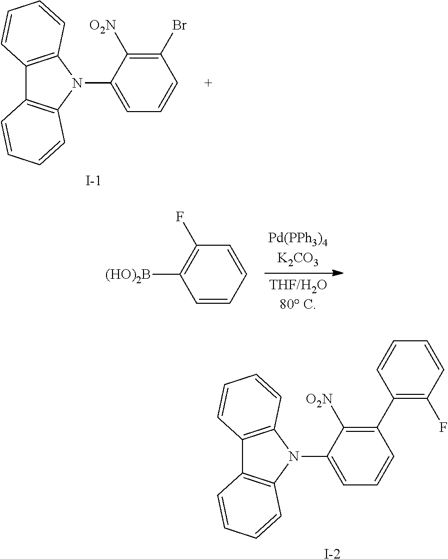

Under a nitrogen atmosphere, Intermediate I-1 (182 g, 496 mmol) was dissolved in 0.8 L of tetrahydrofuran (THF), 2-fluorophenylboronic acid (76.3 g, 545 mmol) purchased from Tokyo Chemical Industry Co., Ltd. and tetrakis(triphenylphosphine) palladium (11.5 g, 9.92 mmol) were added thereto and then, stirred. Subsequently, potassium carbonate (171 g, 1,240 mmol) saturated in water was added thereto and then, heated under reflux at 80° C. for 24 hours. When a reaction was completed, water was added to the reaction solution and then, extracted with dichloromethane (DCM), treated with anhydrous magnesium sulfate to remove moisture, filtered, and concentrated under a reduced pressure. The obtained residue was separated and purified through flash column chromatography, obtaining Intermediate I-2 (95 g, 50%).

HRMS (70 eV, EI+): m/z calcd for C24H15FN2O2: 382.1118, found: 382.

Elemental Analysis: C, 75%; H, 4%

### Synthesis Example 3: Synthesis of Intermediate I-3

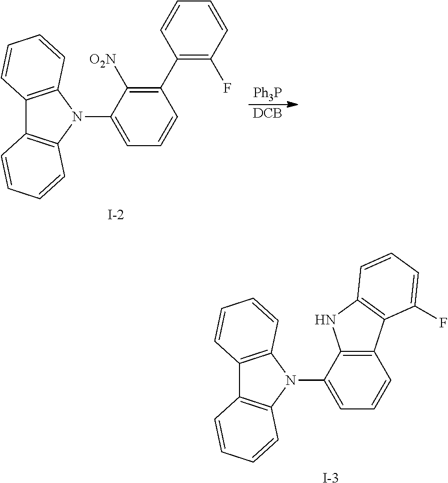

Under a nitrogen atmosphere, Intermediate I-2 (95 g, 248 mmol) was dissolved in 0.1 L of dichlorobenzene (DCB), and triphenylphosphine (195 g, 745 mmol) was added thereto and then, heated under reflux at 200° C. for 7 days. When a reaction was completed, water was added to the reaction solution and then, extracted with dichloromethane (DCM), treated with anhydrous magnesium sulfate to remove moisture, filtered, and concentrated under a reduced pressure. The obtained residue was separated and purified through flash column chromatography, obtaining Intermediate I-3 (36 g, 41%).

HRMS (70 eV, EI+): m/z calcd for C24H15FN2: 350.1219, found: 350.

Elemental Analysis: C, 82%; H, 4%

### Synthesis Example 4: Synthesis of Intermediate I-4

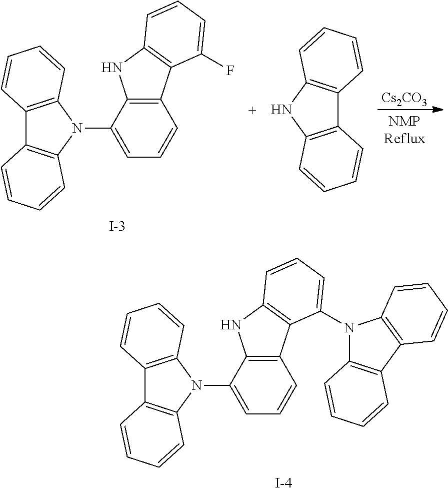

Under a nitrogen atmosphere, Intermediate I-3 (36 g, 94.1 mmol) was dissolved in 0.3 L of N-methyl-2-pyrrolidone (NMP), and 9H-carbazole (31.5 g, 188 mmol) purchased from Tokyo Chemical industry Co., Ltd. and cesium carbonate (61.3 g, 188 mmol) (Mw: 325.82 g/mol, 2 eq) were added thereto and then, heated under reflux for 2 days. When a reaction was completed, after removing the solvent through distillation, water was added to the reaction solution and then, extracted with dichloromethane (DCM), treated with anhydrous magnesium sulfate to remove moisture, filtered, and concentrated under a reduced pressure. The obtained residue was separated and purified through flash column chromatography, obtaining Intermediate I-4 (26 g, 56%).

HRMS (70 eV, EI+): m/z calcd for C36H23N3: 497.1892, found: 497.

Elemental Analysis: C, 87%; H, 5%

### Synthesis Example 5: Synthesis of Compound 1

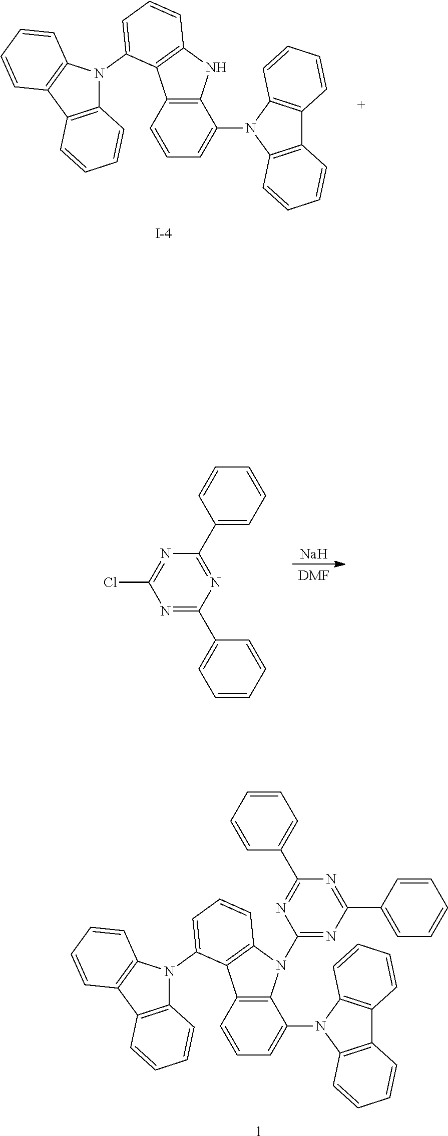

Compound 1 (13.2 g, 90%) was obtained under the same conditions as in Synthesis Example 1 except that Intermediate I-4 (10 g, 20.1 mmol) and 2-chloro-4,6-diphenyl-1,3,5-triazine (6.46 g, 24.1 mmol) purchased from Tokyo Chemical Industry Co., Ltd. were used (e.g., instead of 9H-carbazole and 1-bromo-3-fluoro-2-nitrobenzene).

HRMS (70 eV, EI+): m/z calcd for C51H32N6: 728.2688, found: 728.

Elemental Analysis: C, 84%; H, 4%

### Synthesis Example 6: Synthesis of Compound 2

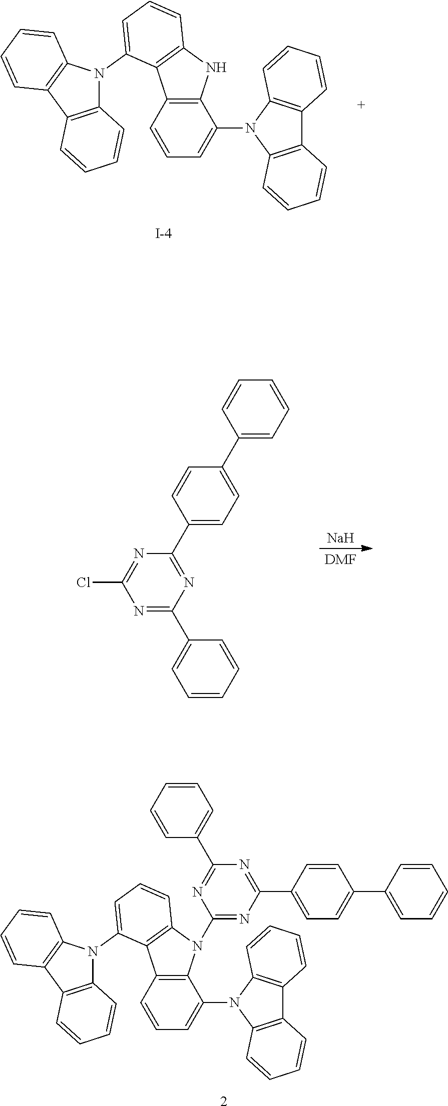

Compound 2 (17.7 g, 88%) was obtained under the same conditions as in Synthesis Example 1 except that Intermediate I-4 (10 g, 20.1 mmol) and 2-(biphenyl-4-yl)-4-chloro-6-phenyl-1,3,5-triazine (8.29 g, 24.1 mmol) purchased from Tokyo Chemical Industry Co., Ltd. were used.

HRMS (70 eV, EI+): m/z calcd for C57H36N6: 804.3001, found: 804.

Elemental Analysis: C, 85%; H, 5%

### Synthesis Example 7: Synthesis of Compound 8

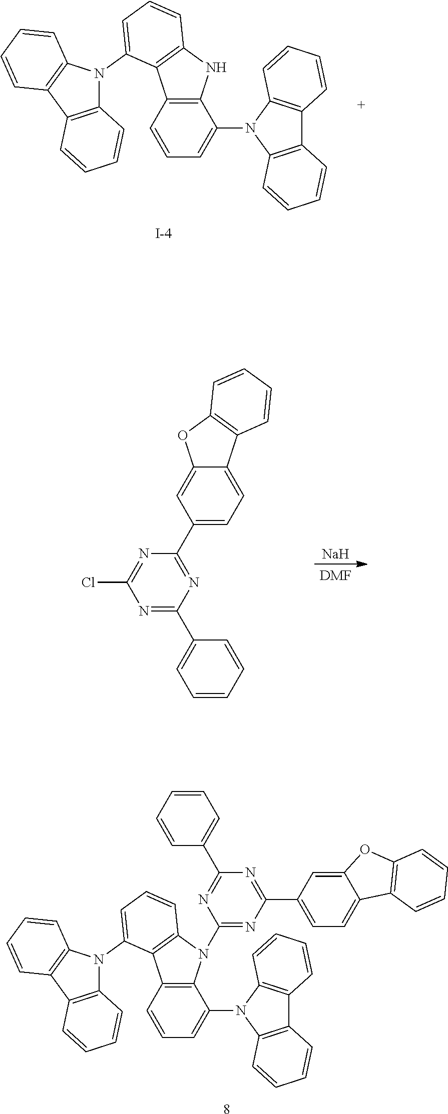

Compound 8 (15.3 g, 93%) was obtained under the same conditions as in Synthesis Example 1 except that Intermediate I-4 (10 g, 20.1 mmol) and 2-chloro-4-(dibenzofuran-3-yl)-6-phenyl-1,3,5-triazine (8.62 g, 24.1 mmol) purchased from P&H Tech Co., Ltd. were used.

HRMS (70 eV, EI+): m/z calcd for C57H34N6O: 818.2794, found: 818.

Elemental Analysis: C, 84%; H, 4%

### Synthesis Example 8: Synthesis of Intermediate I-5

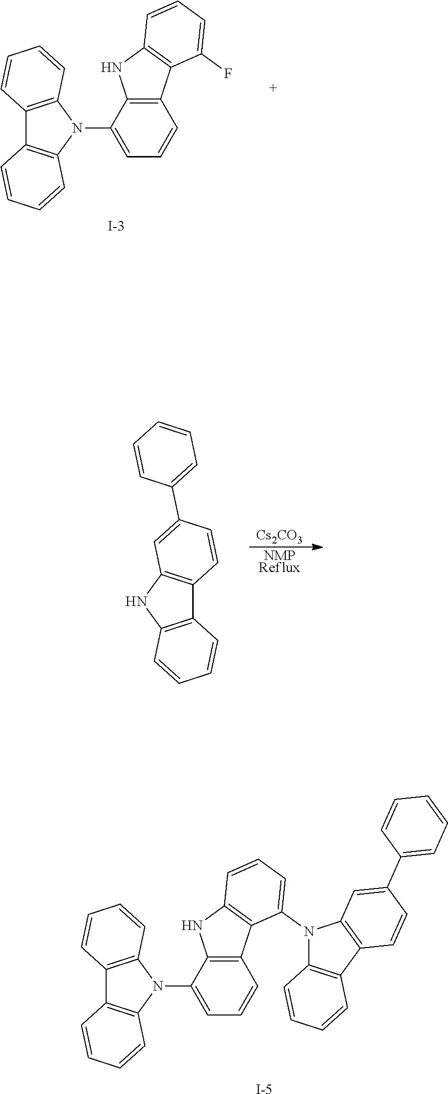

Intermediate I-5 (29.5 g, 60%) was obtained under the same conditions as in Synthesis Example 4 except that Intermediate I-3 (30 g, 85.6 mmol) and 2-phenyl-9H-carbazole (41.7 g, 171 mmol) purchased from Tokyo Chemical Industry Co., Ltd. were used.

HRMS (70 eV, EI+): m/z calcd for C42H27N3: 573.2205, found: 573.

Elemental Analysis: C, 88%; H, 5%

### Synthesis Example 9: Synthesis of Compound 17

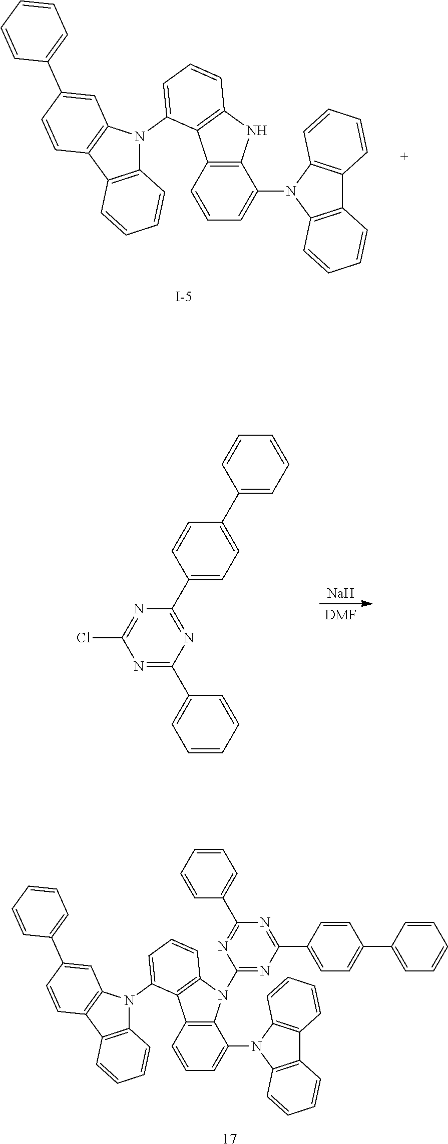

Compound 17 (13.0 g, 85%) was obtained under the same conditions as in Synthesis Example 5 except that Intermediate I-5 (10 g, 17.4 mmol) and 2-(biphenyl-4-yl)-4-chloro-6-phenyl-1,3,5-triazine (7.19 g, 20.9 mmol) purchased from Tokyo Chemical Industry Co., Ltd. were used.

HRMS (70 eV, EI+): m/z calcd for C63H40N6: 880.3314, found: 880.

Elemental Analysis: C, 86%; H, 5%

### Synthesis Example 10: Synthesis of Intermediate I-6

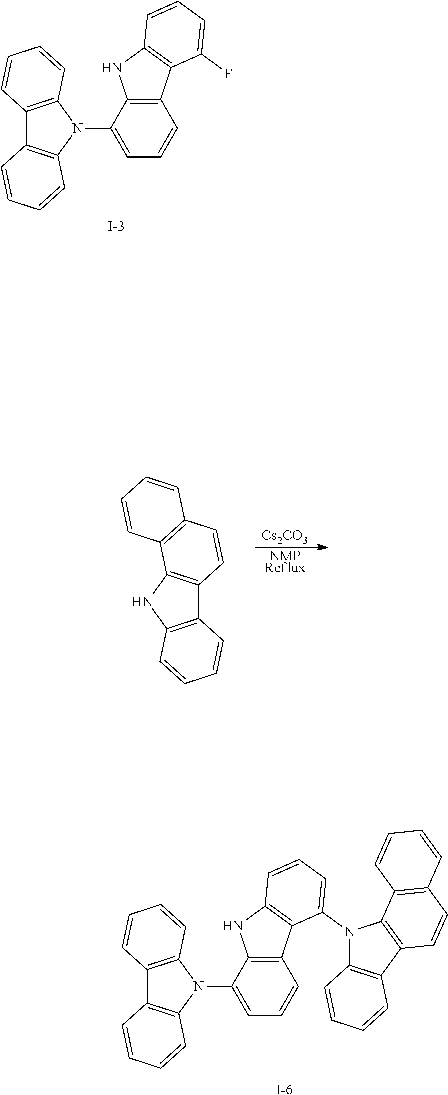

Intermediate I-6 (24.8 g, 53%) was obtained under the same conditions as in Synthesis Example 4 except that Intermediate I-3 (30 g, 85.6 mmol) and 11H-benzo[a]carbazole (37.2 g, 171 mmol) purchased from Tokyo Chemical Industry Co., Ltd. were used.

HRMS (70 eV, EI+): m/z calcd for C40H25N3: 547.2048, found: 547.

Elemental Analysis: C, 88%; H, 5%

### Synthesis Example 11: Synthesis of Compound 25

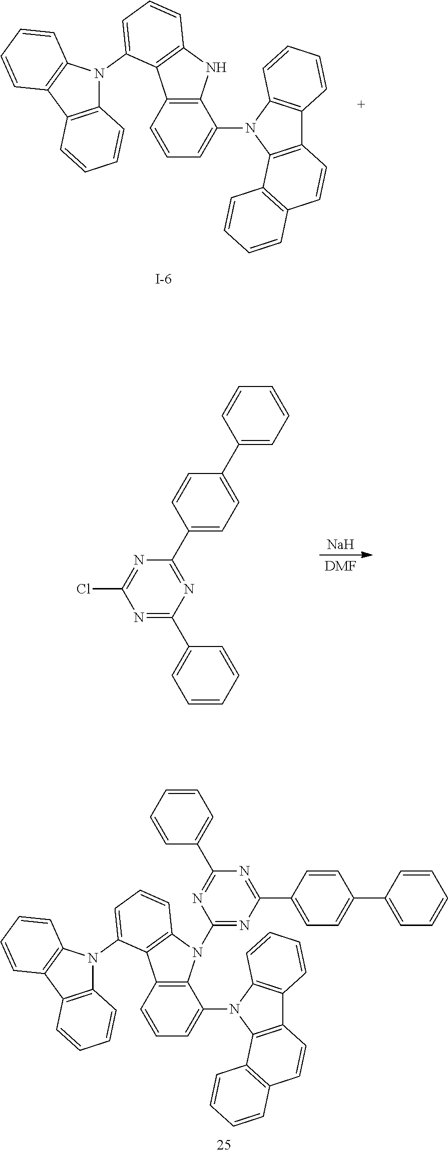

Compound 25 (13.9 g, 89%) was obtained under the same conditions as in Synthesis Example 1 except that Intermediate I-6 (10 g, 18.3 mmol) and 2-(biphenyl-4-yl)-4-chloro-6-phenyl-1,3,5-triazine (7.53 g, 21.9 mmol) purchased from Tokyo Chemical Industry Co., Ltd. were used.

HRMS (70 eV, EI+): m/z calcd for C61H38N6: 854.3158, found: 854.

Elemental Analysis: C, 86%; H, 4%

### Synthesis Example 12: Synthesis of Intermediate I-7

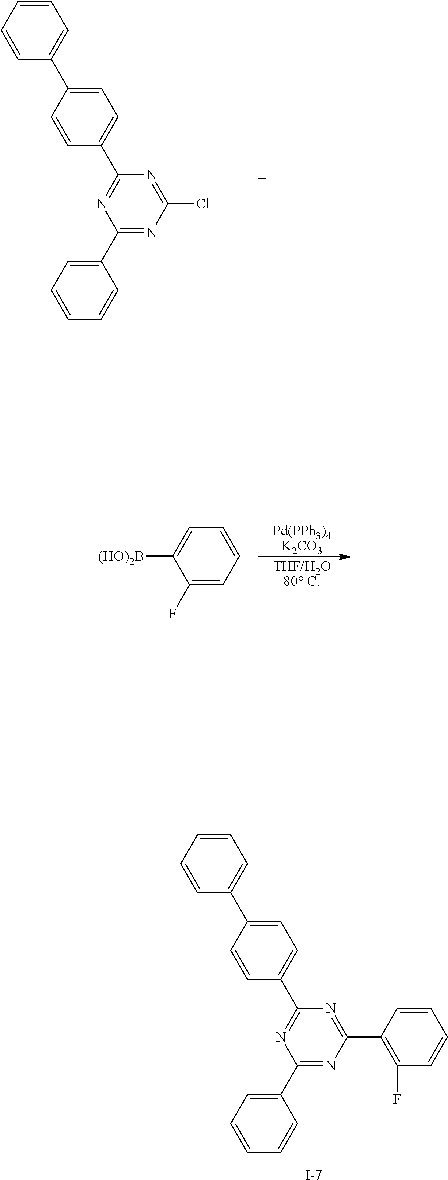

Intermediate I-7 (32.1 g, 91%) was obtained under the same conditions as in Synthesis Example 2 except that 2-(biphenyl-4-yl)-4-chloro-6-phenyl-1,3,5-triazine (30 g, 87.3 mmol) and 2-fluorophenylboronic acid (14.7 g, 105 mmol) purchased from Tokyo Chemical Industry Co., Ltd. were used.

HRMS (70 eV, EI+): m/z calcd for C27H18FN3: 403.1485, found: 403.

Elemental Analysis: C, 80%; H, 5%

### Synthesis Example 13: Synthesis of Compound 36

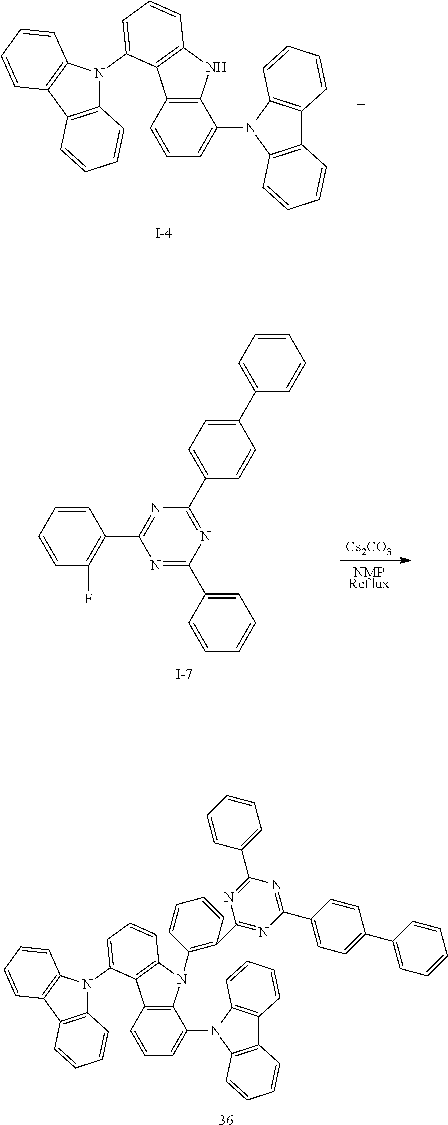

Compound 36 (16.8 g, 95%) was obtained under the same conditions as in Synthesis Example 4 except that Intermediate I-4 (10 g, 20.1 mmol) and Intermediate I-7 (12.2 g, 30.1 mmol) were used.

HRMS (70 eV, EI+): m/z calcd for C63H40N6: 880.3314, found: 880.

Elemental Analysis: C, 86%; H, 5%

### Synthesis Example 14: Synthesis of Intermediate I-8

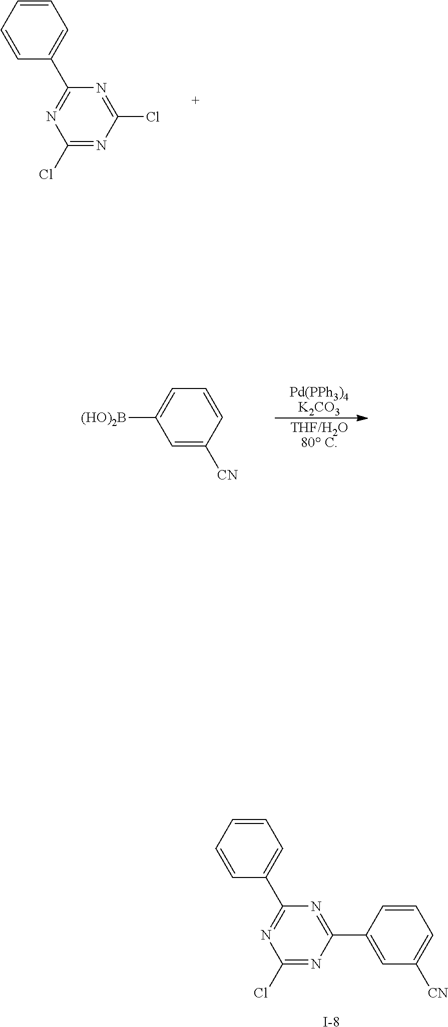

Intermediate I-8 (16.9 g, 62%) was obtained under the same conditions as in Synthesis Example 2 except that 2,4-dichloro-6-phenyl-1,3,5-triazine (30 g, 133 mmol) and 3-cyanophenylboronic acid (13.7 g, 92.9 mmol) purchased from Tokyo Chemical Industry Co., Ltd. were used.

HRMS (70 eV, EI+): m/z calcd for C16H9ClN4: 292.0516, found: 292.

Elemental Analysis: C, 67%; H, 3%

### Synthesis Example 15: Synthesis of Intermediate I-9

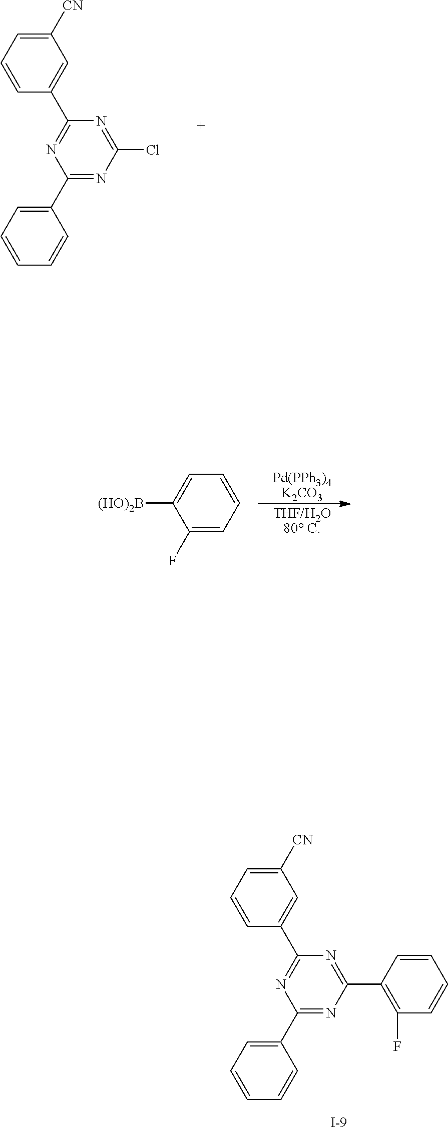

Intermediate I-9 (18.3 g, 95%) was obtained under the same conditions as in Synthesis Example 2 except that Intermediate I-8 (16 g, 54.7 mmol) and 2-fluorophenylboronic acid (9.18 g, 65.6 mmol) purchased from Tokyo Chemical Industry Co., Ltd. were used.

HRMS (70 eV, EI+): m/z calcd for C22H13FN4: 352.1124, found: 352.

Elemental Analysis: C, 75%; H, 4%

### Synthesis Example 16: Synthesis of Compound 69

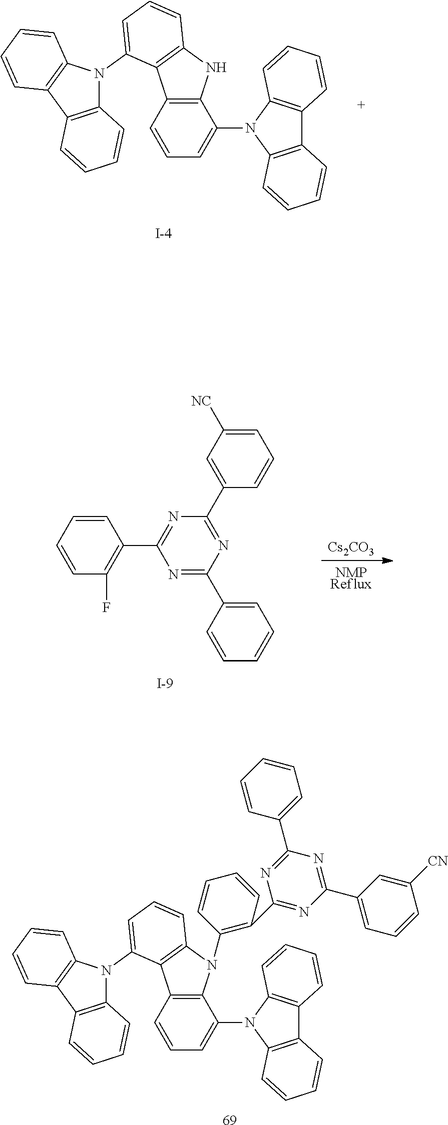

Compound 69 (14.0 g, 84%) was obtained under the same conditions as in Synthesis Example 4 except that Intermediate I-4 (10 g, 20.1 mmol) and Intermediate I-9 (9 g, 25.5 mmol) were used.

HRMS (70 eV, EI+): m/z calcd for C58H35N7: 829.2954, found: 829.

Elemental Analysis: C, 84%; H, 4%

### Synthesis Example 17: Synthesis of Host 1

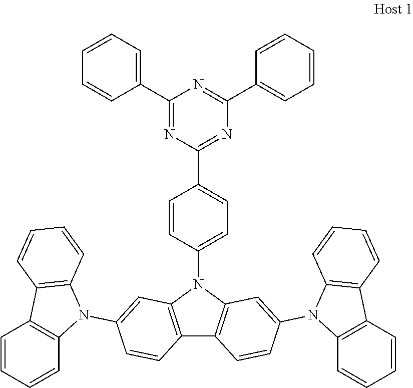 [SMILES: c1ccc(-c2nc(-c3ccccc3)nc(-c3ccc(-n4c5cc(-n6c7ccccc7c7ccccc76)ccc5c5ccc(-n6c7ccccc7c7ccccc76)cc54)cc3)n2)cc1]

Host 1 was synthesized with reference to the synthesis method of Korean patent KR 2019-0043840.

HRMS (70 eV, EI+): m/z calcd for C57H36N6: 804.3001, found: 804.

Elemental Analysis: C, 85%; H, 5%

### Synthesis Example 18: Synthesis of Host 2

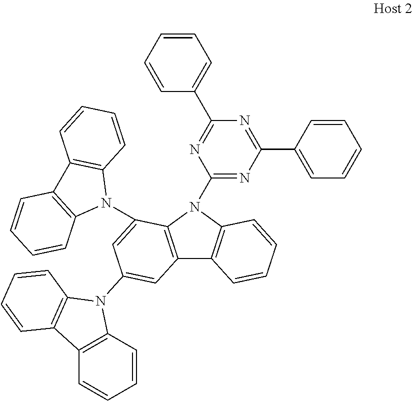 [SMILES: c1ccc(-c2nc(-c3ccccc3)nc(-n3c4ccccc4c4cc(-n5c6ccccc6c6ccccc65)cc(-n5c6ccccc6c6ccccc65)c43)n2)cc1]

Host 2 was synthesized with reference to the synthesis method of Korean patent KR 2011-0043342.

HRMS (70 eV, EI+): m/z calcd for C51H32N6: 728.2688, found: 728.

Elemental Analysis: C, 84%; H, 4%

### Synthesis Example 19: Synthesis of Host 3

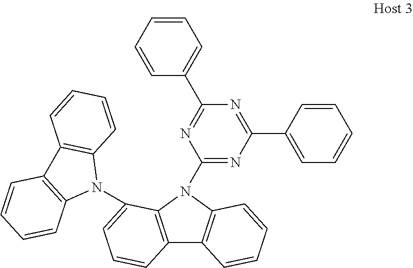 [SMILES: c1ccc(-c2nc(-c3ccccc3)nc(-n3c4ccccc4c4cccc(-n5c6ccccc6c6ccccc65)c43)n2)cc1]

Host 3 was synthesized with reference to the synthesis method of Korean patent KR 2020-0078254.

HRMS (70 eV, EI+): m/z calcd for C39H25N5: 563.2110, found: 563.

Elemental Analysis: C, 83%; H, 4%

### Synthesis Example 20: Synthesis of Intermediate I-10

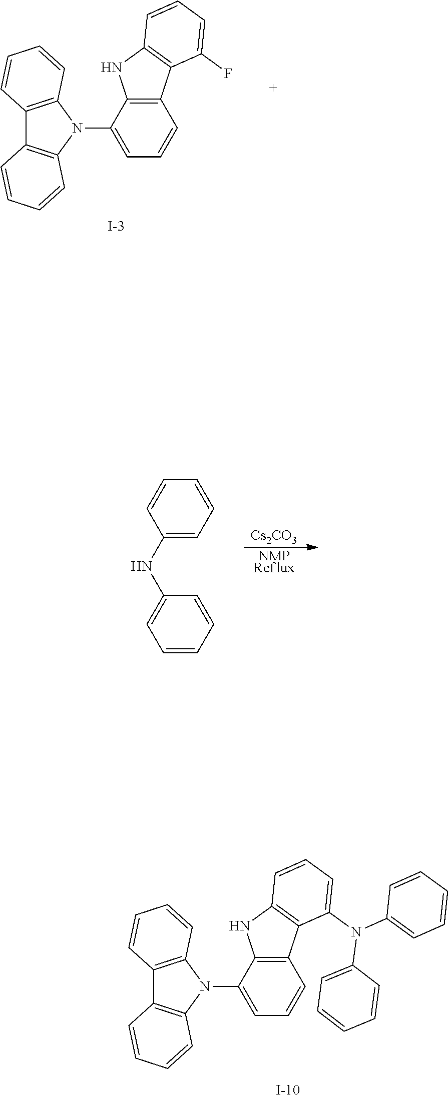

Intermediate I-10 (21.4 g, 50%) was obtained under the same conditions as in Synthesis Example 4 except that Intermediate I-3 (30 g, 85.6 mmol) and diphenylamine (28.9 g, 171 mmol) purchased from Sigma Aldrich were used.

HRMS (70 eV, EI+): m/z calcd for C36H25N3: 499.2048, found: 499.

Elemental Analysis: C, 87%; H, 5%

### Synthesis Example 21: Synthesis of Compound Host 4

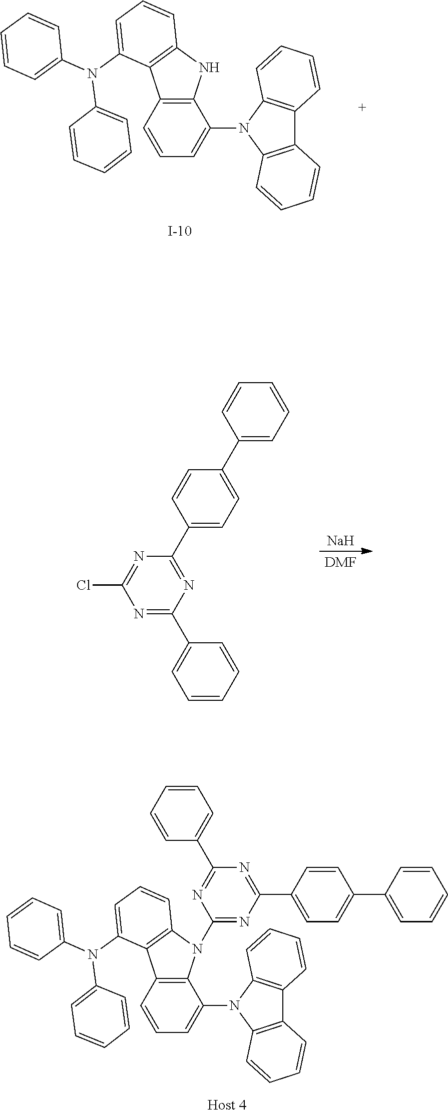

Host 4 (13.4 g, 83%) was obtained under the same conditions as in Synthesis Example 1 except that Intermediate I-10 (10 g, 20 mmol) and 2-(biphenyl-4-yl)-4-chloro-6-phenyl-1,3,5-triazine (8.26 g, 24.0 mmol) purchased from Tokyo Chemical Industry were used.

HRMS (70 eV, EI+): m/z calcd for C57H38N6: 806.3158, found: 806.

Elemental Analysis: C, 85%; H, 5%

Manufacture of Organic Light Emitting Diode (Green)

### Example 1

An organic light emitting diode was manufactured using Compound 1 as a host and Ir(PPy)₃ as a dopant. The anode included ITO having a thickness of 1,000 Å, and the cathode included aluminum (Al) having a thickness of 1,000 Å. Specifically, an ITO glass substrate having a sheet resistance of 15 Ω/cm² was cut into a size of 50 mm×50 mm×0.7 mm, and was ultrasonically cleaned for 15 minutes in acetone, isopropyl alcohol, and pure water, and after ultrasonic cleaning, UV ozone cleaned for 30 minutes, and the resultant was used as an anode. N4,N4′-di(naphthalen-1-yl)-N4,N4′-diphenylbiphenyl-4,4′-diamine (NPB) was deposited under the condition of a vacuum degree of 650×10⁻⁷ Pa and a deposition rate of 0.1 to 0.3 nm/s on the substrate to form a hole transport layer having a thickness of 800 Å. Subsequently, a light emitting layer having a thickness of 300 Å was formed using Compound 1 under the same vacuum deposition conditions, and at this time, Ir(PPy)₃ as a phosphorescent dopant was simultaneously deposited. At this time, by controlling the deposition rate of the phosphorescent dopant, when the total amount of the light emitting layer was 100 wt %, the phosphorescent dopant was deposited so that the amount was 7 wt %. Bis(2-methyl-8-quinolinolate)-4-(phenylphenolato)aluminium (BAlq) was deposited on the light emitting layer under the same vacuum deposition conditions to form a hole blocking layer having a thickness of 50 Å. Then, Alq₃ was deposited under the same vacuum deposition conditions to form an electron transport layer having a film thickness of 200 Å. An organic light emitting diode was manufactured by sequentially depositing LiF and Al as a cathode on the electron transport layer.

The organic light emitting diode had a structure of ITO/NPB (80 nm)/EML (Compound 1 (93 wt %)+Ir(PPy)₃ (7 wt %), 30 nm)/BAlq (5 nm)/Alq₃ (20 nm)/LiF (1 nm)/Al (100 nm).

The structures of NPB, BAlq, CBP, and Ir(PPy)₃ used to manufacture the organic light emitting diode are as follows.

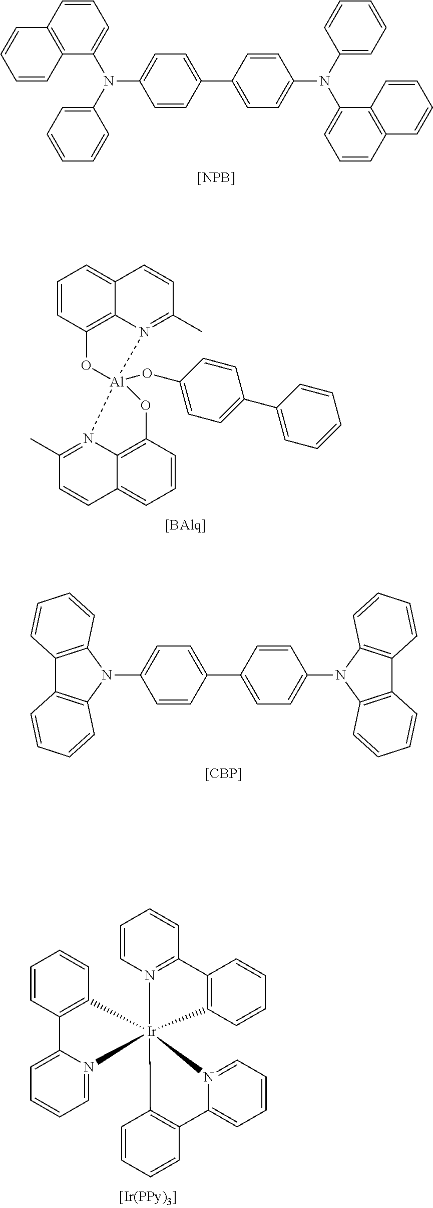

### Examples 2 to 7 and Comparative Examples 1 to 5

Organic light emitting diodes were manufactured in the same manner as in Example 1, except that the compositions shown in Table 1 were used, instead of Compound 1.

Evaluation

Current density change, luminance change, and luminous efficiency according to voltage of the organic light emitting diodes according to Examples 1 to 7 and Comparative Examples 1 to 5 were measured.

Specific measurement methods are as follows, and the results are shown in Table 1.

(1) Measurement of Current Density Change Depending on Voltage Change

The manufactured organic light emitting diodes were measured regarding a current value flowing in the unit device, while increasing the voltage from 0 V to 10 V using a current-voltage meter (Keithley 2400), and the measured current value is divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

The luminous efficiency (cd/A) of the same current density (10 mA/cm²) was calculated using the luminance and current density from the items (1) and (2) and voltages.

(4) Measurement of Life-Span

The luminance (cd/m²) to be maintained at 5,000 cd/m² and the time when current efficiency (cd/A) is decreased to 90%, to obtain the results.

(5) Driving Voltage

The driving voltage of each diode at 15 mA/cm² was measured using a current-voltmeter (Keithley 2400), and the results were obtained.

TABLE 1

| | | Driving | Efficiency | 90% life-span (h) |
|---|---|---|---|---|
| No. | Compound | voltage (V) | (cd/A) | at 5,000 cd/m² |
| Example 1 | 1 | 3.95 | 60.0 | 1,050 |
| Example 2 | 2 | 3.85 | 63.5 | 1,250 |
| Example 3 | 8 | 3.80 | 62.0 | 1,300 |
| Example 4 | 17 | 3.85 | 63.0 | 1,450 |
| Example 5 | 25 | 3.90 | 58.0 | 1,000 |
| Example 6 | 36 | 3.90 | 65.5 | 1,500 |
| Example 7 | 69 | 3.85 | 59.0 | 1,600 |
| Comparative | CBP | 4.81 | 31.4 | 40 |
| Example 1 | | | | |
| Comparative | Host 1 | 4.05 | 55.0 | 700 |
| Example 2 | | | | |
| Comparative | Host 2 | 4.00 | 56.0 | 850 |
| Example 3 | | | | |
| Comparative | Host 3 | 4.20 | 53.5 | 450 |
| Example 4 | | | | |
| Comparative | Host 4 | 4.00 | 55.5 | 350 |
| Example 5 | | | | |

Referring to Table 1, the organic light emitting diodes according to Examples 1 to 7 exhibited a low driving voltage, high efficiency, and a long life-span, compared to the organic light emitting diodes according to Comparative Examples 1 to 5.

One or more embodiments may provide a compound for an organic optoelectronic device capable of realizing a high-efficiency and long life-span organic optoelectronic device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 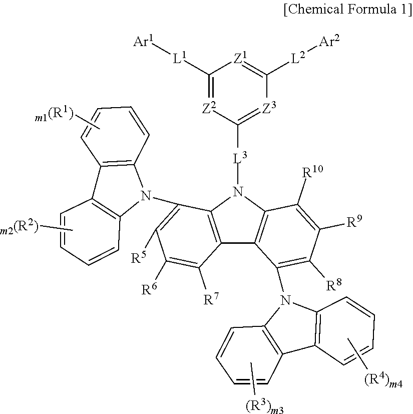
wherein, in Chemical Formula 1,
Z¹ to Z³ are each independently N or CR^{a}, at least two of Z¹ to Z³ being N,
L¹ and L² are each independently a single bond, a substituted or unsubstituted C6 to C30 arylene group, or a substituted or unsubstituted C2 to C30 heterocyclic group,
L³ is a single bond or a substituted or unsubstituted C6 to C30 arylene group,
Ar¹ and Ar² are each independently a substituted or unsubstituted C6 to C30 aryl group or a substituted or unsubstituted C2 to C30 heterocyclic group,
R^{a} and R¹ to R¹⁰ are each independently hydrogen, deuterium, a substituted or unsubstituted C1 to C20 alkyl group, or a substituted or unsubstituted C6 to C20 aryl group, and
m1 to m4 are each independently an integer of 1 to 4.

2. The compound as claimed in claim 1, wherein L³ is a single bond or a substituted or unsubstituted phenylene group.

3. The compound as claimed in claim 1, wherein L³ is a substituted or unsubstituted ortho-phenylene group.

4. The compound as claimed in claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted fluorenyl group, a substituted or unsubstituted dibenzosilolyl group, a substituted or unsubstituted dibenzofuranyl group, a substituted or unsubstituted dibenzothiophenyl group, or a substituted or unsubstituted carbazolyl group.

5. The compound as claimed in claim 1, wherein L¹ and L² are each independently a single bond, a substituted or unsubstituted phenylene group, or a substituted or unsubstituted naphthylene group.

6. The compound as claimed in claim 1, wherein:
moieties *-L¹-Ar¹ and *-L²-Ar² are each independently a moiety of Group I: 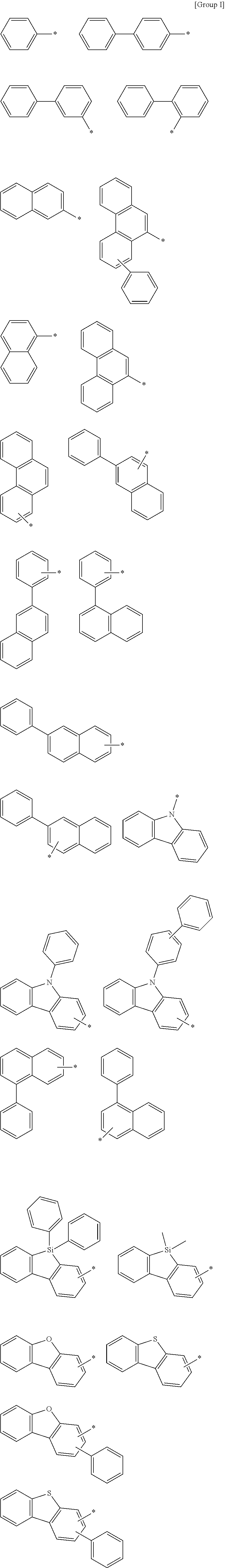
in Group I, * is a linking point.

7. The compound as claimed in claim 1, wherein the compound is a compound of Group 1: 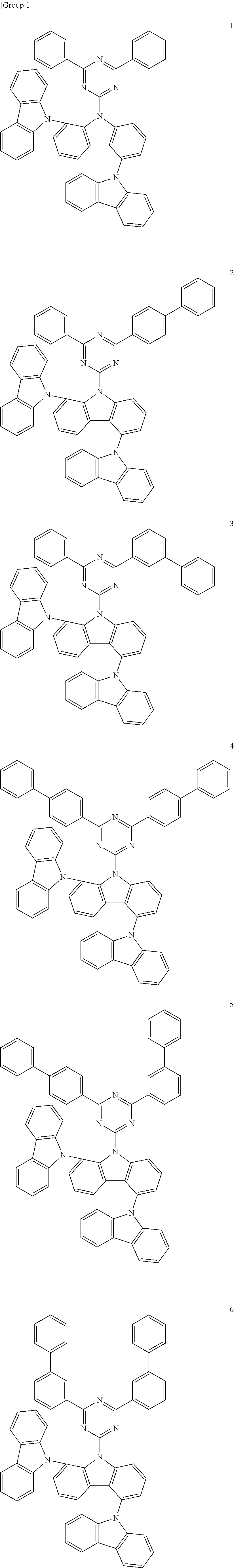 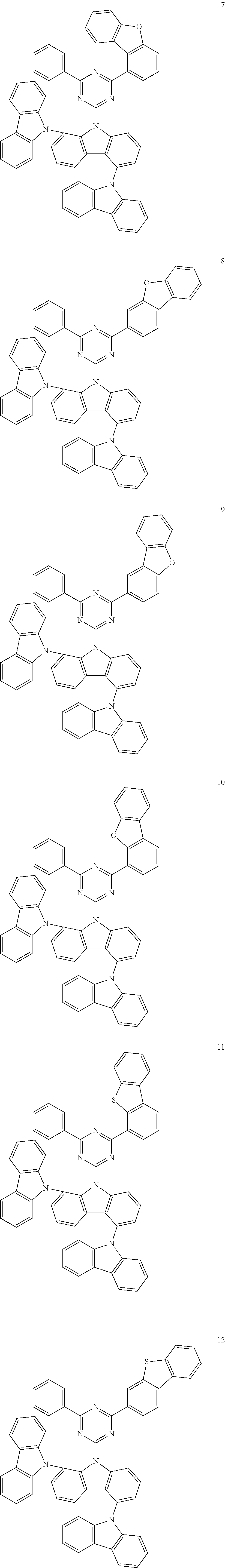 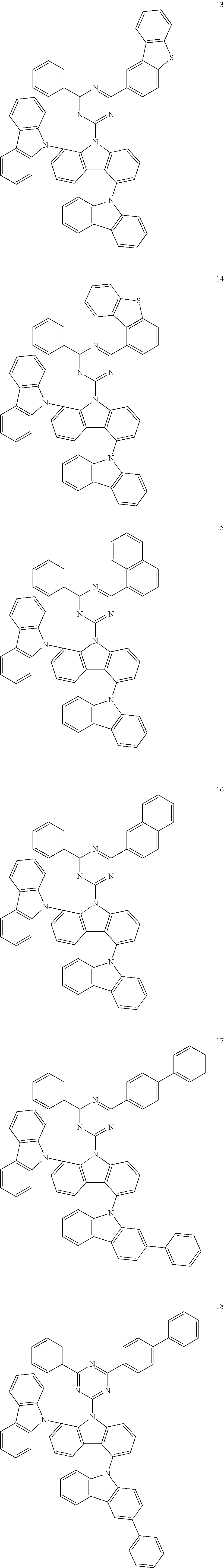 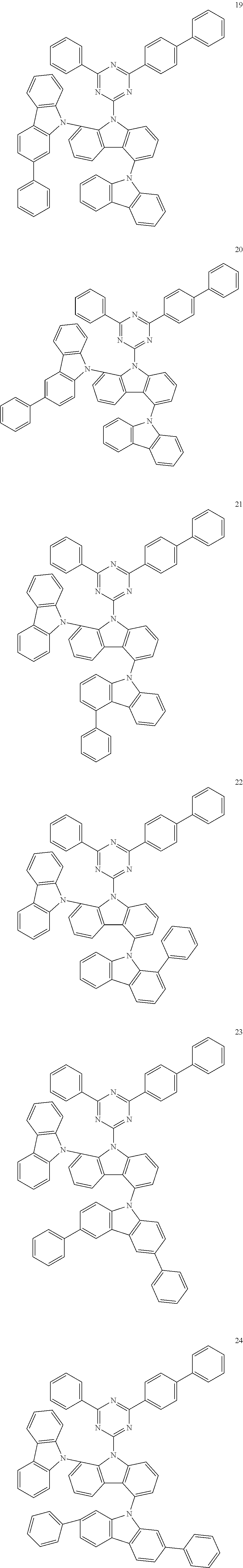 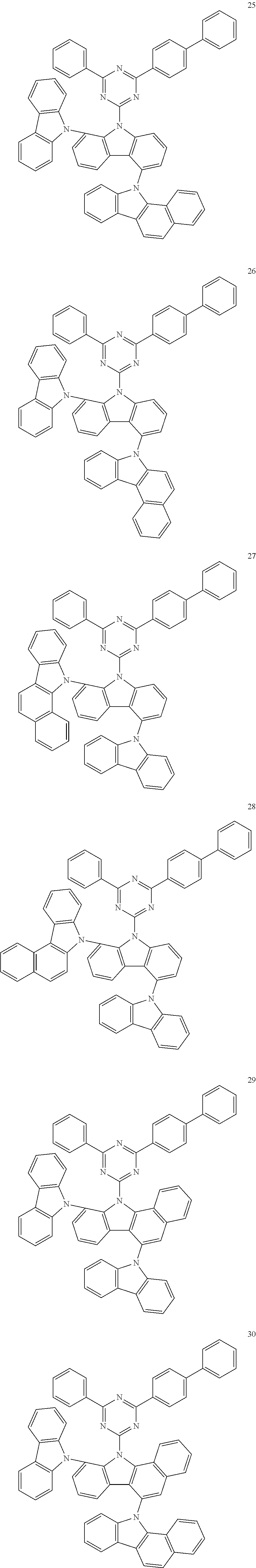 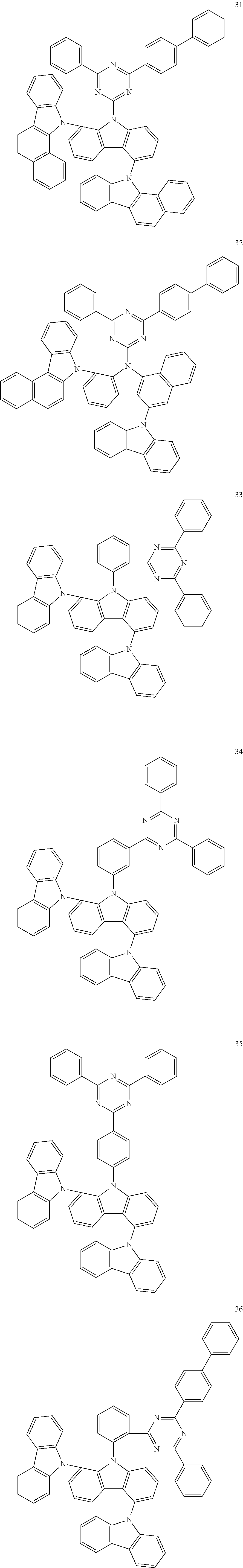 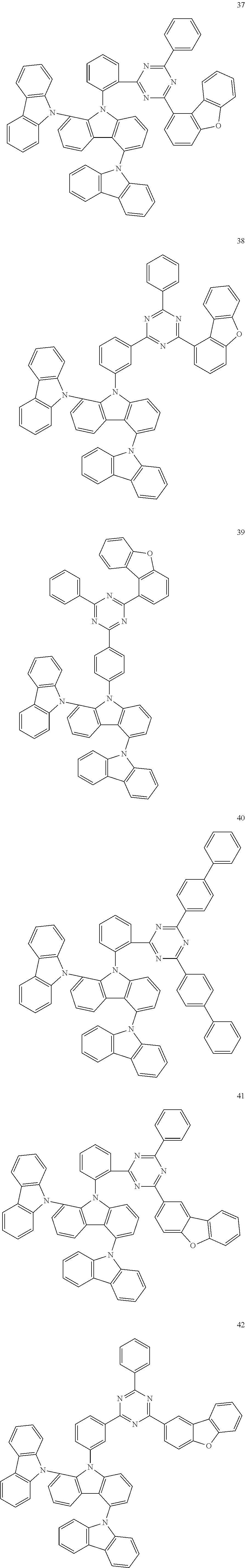 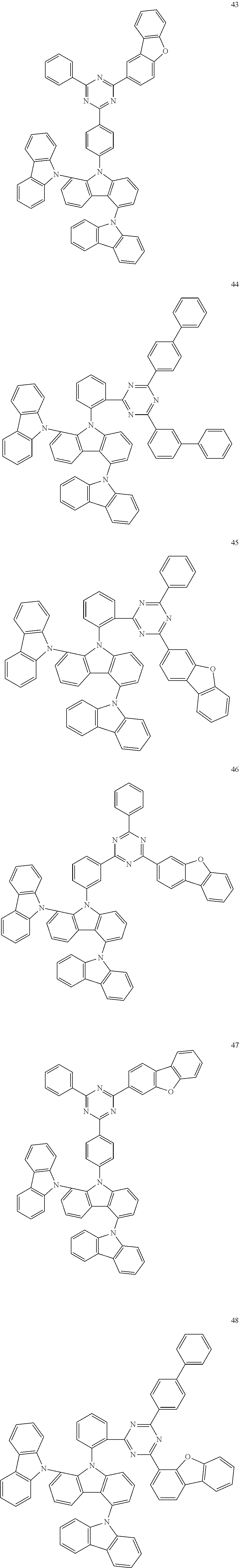 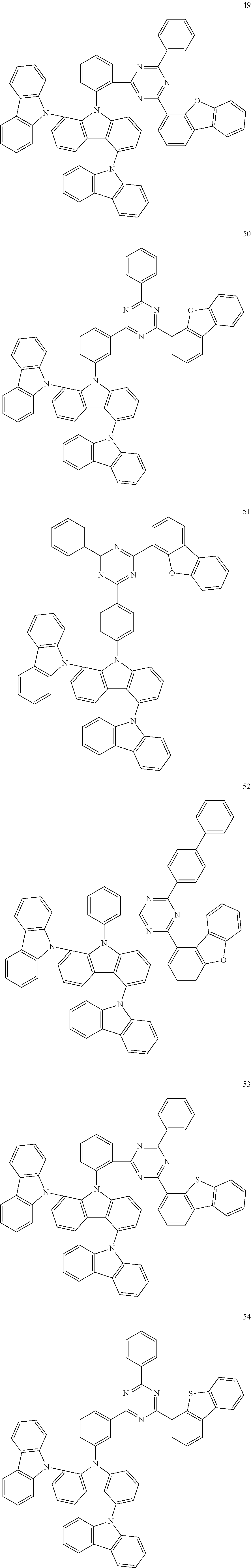 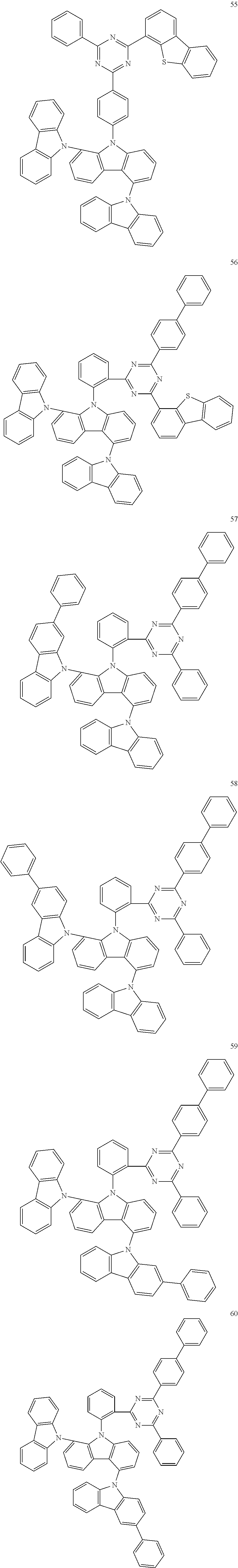 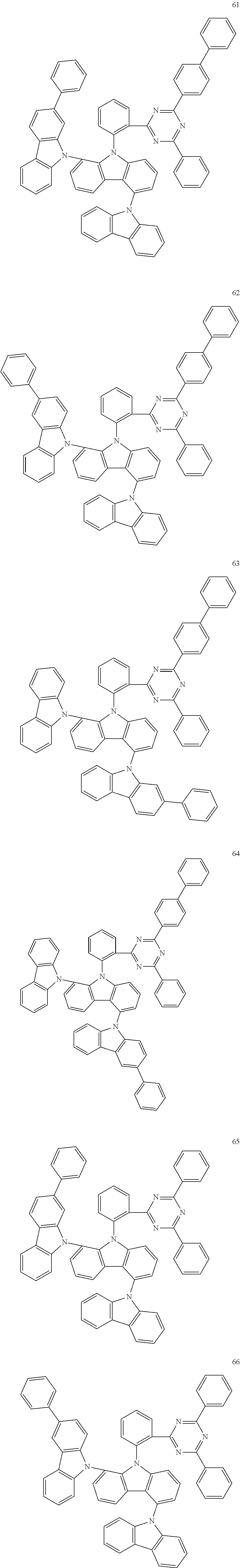 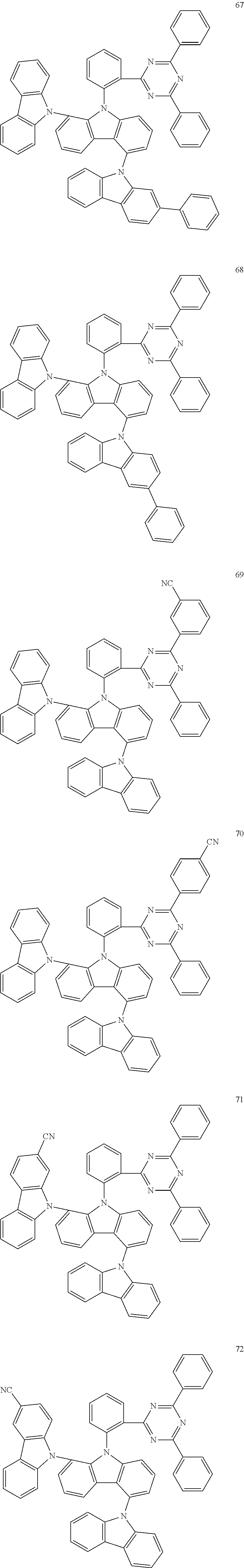 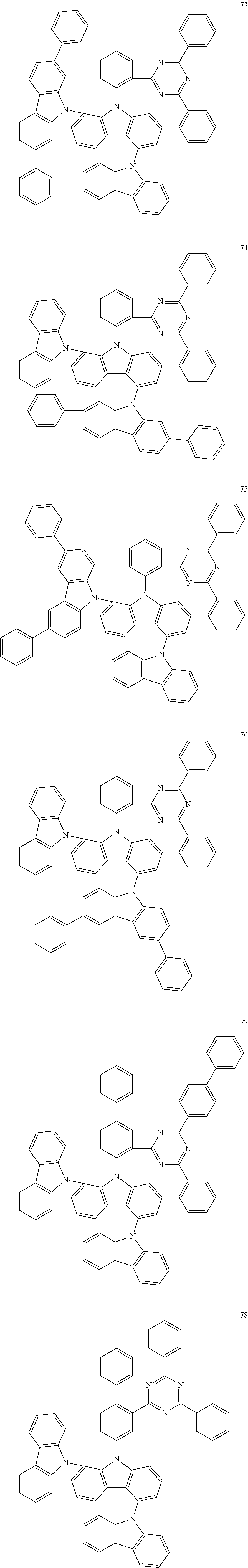 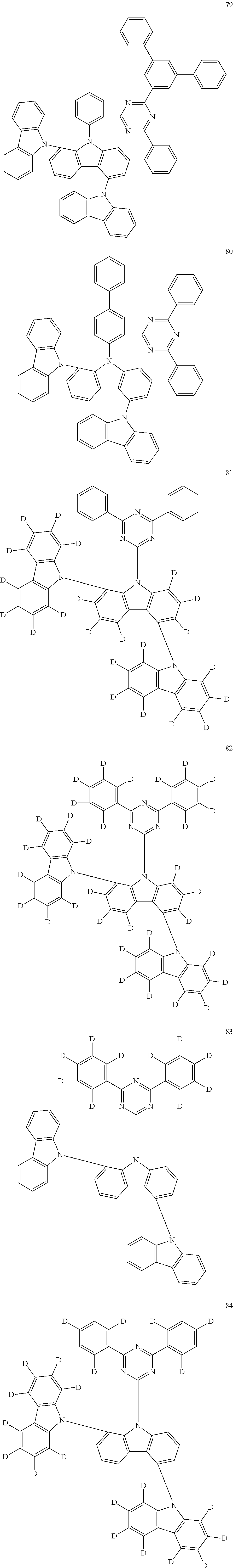
wherein D is deuterium.

8. An organic optoelectronic device, comprising:
an anode and a cathode facing each other, and
at least one organic layer between the anode and the cathode,
wherein the at least one organic layer includes a light emitting layer, and
the light emitting layer includes the compound as claimed in claim 1.

9. The organic optoelectronic device as claimed in claim 8, wherein the compound is a host of the light emitting layer.

10. A display device comprising the organic optoelectronic device as claimed in claim 8.
